(19) United States
(12) Patent Application Publication
(10) Pub. No.: US 2021/0395679 A1
(43) Pub. Date: Dec. 23, 2021

(54) IN VITRO CELL CULTURE SYSTEM FOR PRODUCING HEPATOCYTE-LIKE CELLS AND USES THEREOF

(71) Applicant: CHILDREN'S HOSPITAL MEDICAL CENTER, Cincinnati, OH (US)

(72) Inventor: Akihiro ASAI, Cincinnati, OH (US)

(21) Appl. No.: 17/292,162

(22) PCT Filed: Nov. 8, 2019
(86) PCT No.: PCT/US2019/060605

§ 371 Date: May 7, 2021

Related U.S. Application Data
(60) Provisional application No. 62/757,799, filed on Nov. 9, 2018.

(51) Int. Cl. C12N 5/071; C12N 1/02; C12M 1/12; C12M 1/00

(52) U.S. Cl. CPC C12N 5/067; C12N 1/02; C12M 25/02; C12M 23/34; C12N 2501/115; C12N 2501/12; C12N 2533/90; C12N 2501/16; C12N 2501/415; C12N 2510/00; C12N 2506/45; C12N 2501/33; C12N 2501/155

(57) ABSTRACT

The present disclosure provides methods for generating an in vitro model of cholestatic liver disease and uses of the same. In some embodiments, the methods involve an in vitro culture system for producing hepatocyte-like cells from pluripotent stem cells.

### CROSS-REFERENCE TO RELATED APPLICATIONS

This application claims the benefit of the filing date of U.S. Provisional Application No. 62/757,799, filed Nov. 9, 2018, the entire contents of which are incorporated by reference herein.

### INCORPORATION OF SEQUENCE LISTING

A computer readable text file, entitled “103144-637732-70037WO00-Seq-Listing.txt” created on or about Nov. 8, 2019, with a file size of about 1 KB, contains the sequence listing for this application and is hereby incorporated by reference in its entirety.

### BACKGROUND OF THE INVENTION

Cholestasis is defined as a decrease in bile flow due to impaired secretion by hepatocytes or to obstruction of bile flow through intra- or extrahepatic bile ducts. Therefore, the clinical definition of cholestasis is any condition in which substances normally excreted into bile are retained. The serum concentrations of conjugated bilirubin and bile salts are the most commonly measured.

Bile acids, the major component of bile, are cholesterol metabolites that are formed in the liver and secreted into the duodenum of the intestine, where they have important roles in the solubilization and absorption of dietary lipids and vitamins. Most bile acids (˜95%) are subsequently reabsorbed in the ileum and returned to the liver via the enterohepatic circulatory system. Hepato-enteric recirculation of bile acids regulates a balance between de novo synthesis and sinusoid-to-canalicular transport of bile acids in hepatocytes. This is mediated by the intracellular accumulation of bile acids. Since bile flow is dependent on efficient bile acid transport by hepatocytes, genetic defects affecting bile acid transporters, which disturb the canalicular export of bile acids and result in cholestasis. The characteristic pattern of clinical presentation includes jaundice, pruritus, elevated serum bile acid levels, fat malabsorption, fat soluble vitamin deficiency, and liver injury.

Cholestasis often does not respond to medical therapy of any sort. Some reports indicate success in children with chronic cholestatic diseases with the use of ursodeoxycholic acid, which acts to increase bile formation and antagonizes the effect of hydrophobic bile acids on biological membranes. Phenobarbital may also be useful in some children with chronic cholestasis.

Treatment of fat malabsorption principally involves dietary substitution. In older patients, a diet that is rich in carbohydrates and proteins can be substituted for a diet containing long-chain triglycerides. In infants, that may not be possible, and substitution of a formula containing medium-chain triglycerides may improve fat absorption and nutrition.

In chronic cholestasis, careful attention must be paid to prevent fat-soluble vitamin deficiencies, which are common complications in pediatric patients with chronic cholestasis. This is accomplished by administering fat-soluble vitamins and monitoring the response to therapy. Oral absorbable, fat-soluble vitamin formulation A, D, E, and K supplementation is safe and potentially effective in pediatric patients with cholestasis.

It is therefore of great interest to develop new models to gain a greater understanding of the pathogenic mechanisms of cholestatic liver diseases, to provide insight into therapeutic targeting in subjects suffering from cholestasis, and to screen for drug candidate for treating the disease.

### SUMMARY OF THE INVENTION

The present disclosure is based unexpected discovery of an in vitro disease model for genetic cholestatic liver disease as disclosed herein, which form apico-basolateral polarity needed to investigate bile acid transport in hepatocytes while recapitulating hepatocyte disease pathologies. The novel in vitro model can help provide new insights into molecular mechanisms that underlie the pathophysiology of cholestatic liver disease, a model for screening therapeutic agents and provide targets for therapeutic intervention in patients.

Accordingly, one aspect of the present disclosure features a method for generating a population of hepatocyte-like cells from a population of pluripotent stem cells. In some examples, the pluripotent stem cells can be induced pluripotent stem cells (iPSCs).

In some embodiments, the method disclosed herein may comprise: (i) culturing a population of pluripotent stem cells in an endoderm differentiation medium; wherein the pluripotent stem cells comprise a genetically modified ABCB11 gene; (ii) culturing a population of cells obtained from step (i) in a hepatic specification medium; and (iii) culturing a population of cells obtained from step (ii) in a hepatocyte maturation medium to produce a population of hepatocyte-like cells. In some examples, step (iii) may be performed in the absence of human umbilical vein endothelial cells (HUVEC) and/or mesenchymal stem cells (MSC) to produce a population of hepatocyte-like cells.

In some instances, the genetically modified ABCB11 gene expresses a truncated mutant of a bile salt export pump (BSEP) protein. Examples include a R1090X truncation mutant. In some instances, the genetic modification of the ABCB11 gene is performed by CRISPR/Cas9-mediated gene editing.

In other embodiments, the method of generating a population of hepatocyte-like cells provided herein may comprise: (i) culturing a population of pluripotent stem cells in an endoderm differentiation medium; (ii) culturing a population of cells obtained from step (i) in a hepatic specification medium; and (iii) culturing a population of cells obtained from step (ii) in a hepatocyte maturation medium, wherein step (iii) is performed in the absence of human umbilical vein endothelial cells (HUVEC) and/or mesenchymal stem cells (MSC) to produce a population of hepatocyte-like cells.

In any of the methods disclosed herein the endoderm differentiation medium may comprise: (a) an activin, (b) insulin, and (c) an inhibitor of class I histone deacetylase, an activator of Wnt signaling pathway, a Rho-associated protein kinase (ROCK) inhibitor, a GSK3 inhibitor, or a combination thereof. In some examples, the endoderm differentiation medium may comprise an activin, insulin, the activator of Wnt signaling pathway, and the ROCK inhibitor. In some examples, the endoderm differentiation medium may comprise an activin, insulin, the activator of Wnt signaling pathway, and the inhibitor of class I histone deacetylase. In other examples, the endoderm differentiation medium may comprise an activin, insulin, the GSK3 inhibitor, and the inhibitor of class I histone deacetylase. In yet other examples, the endoderm differentiation medium may comprise an activin, insulin, the GSK3 inhibitor, and the ROCK inhibitor. In further examples, the endoderm differentiation medium may comprise an activin, insulin, and the GSK3 inhibitor.

In any of the methods disclosed herein, the inhibitor of class I histone deacetylase may be sodium butyrate; the activator of Wnt signaling pathway may be Wnt3a; the GSK inhibitor may be CHIR99021, and/or the ROCK inhibitor is Y 27632.

In some examples, step (i) of any of the method disclosed herein may be performed by culturing the population of pluripotent stem cells in the endoderm differentiation medium for about 5-8 days. In one specific example, step (i) can be performed by (a) culturing the population of pluripotent stem cells in a first endoderm differentiation medium for one day, wherein the first endoderm differentiation medium comprises an activin, insulin, the activator of Wnt signaling pathway, and the ROCK inhibitor; (b) culturing the population of pluripotent stem cells in a second endoderm differentiation medium following step (a) for one day, wherein the second endoderm differentiation medium comprises an activin, insulin, the activator of Wnt signaling pathway, and the inhibitor of class I histone deacetylase; (c) culturing the population of pluripotent stem cells in a third endoderm differentiation medium following step (c) for two days, wherein the third endoderm differentiation medium comprises an activin, insulin, the GSK3 inhibitor, and the inhibitor of class I histone deacetylase; (d) culturing the population of pluripotent stem cells in a fourth endoderm differentiation medium following step (c) for one day, wherein the fourth endoderm differentiation medium comprises an activin, insulin, the GSK3 inhibitor, and the ROCK inhibitor; and (e) culturing the population of pluripotent stem cells in a fifth endoderm differentiation medium following step (d) for one day, wherein the fifth endoderm differentiation medium comprises an activin, insulin, and the GSK3 inhibitor. In some instances, after step (c) and prior to step (d), the population of pluripotent stem cells can be placed on a permeable membrane.

In some examples, step (i) may comprise culturing the cells in a first cell culture vessel comprising an upper chamber and a lower chamber; wherein both the upper chamber and the lower chamber are separated with a permeable membrane optionally coated with at least one extracellular matrix protein and wherein the cells are in contact with the permeable membrane. For example, the cells can be first cultured in a second cell culture vessel for about 4 days and then cultured in the first cell culture vessel. In some instances, the first culture vessel, the second culture vessel, or both are coated with at least one extracellular matrix protein. In some instances, the inhibitor of class I deacetylase activity can be removed from the medium after about 3 days.

In any of the methods disclosed herein, the hepatic specification medium may comprise: (a) a fibroblast growth factor (FGF), and (b) a bone morphogenic protein (BMP). In some examples, the FGF can be FGF2 and/or the BMP can be BMP4. Step (ii) may be performed by culturing the population of cells from step (i) in the hepatic specification medium for about 3 days.

In any of the methods disclosed herein, the hepatocyte maturation medium may comprise a hepatocyte growth factor (HGF) and is free of a human epidermal growth factor (EGF). In some embodiments, the hepatocyte maturation medium may further comprise transferrin, hydrocortisone, and insulin.

In some embodiments, step (iii) may comprise culturing the population of cells from step (ii) on a permeable membrane in a cell culture vessel. Such a cell culture vessel may comprise an upper chamber and a lower chamber; wherein both the upper chamber and the lower chamber are separated with the permeable membrane and wherein the cells are placed on the permeable membrane. In some instances, the permeable membrane is coated with at least one extracellular matrix protein. In some instances, step (iii) can be performed by culturing the population of cells from step (ii) for about 10-14 days.

Also provided herein are hepatocyte-like cells, produced by any of the methods disclosed herein. Such hepatocyte-like cells form apico-basolateral polarity.

In another aspect, provided herein is an in vitro cell culture system, comprising: (i) a cell culture vessel comprising an upper chamber and a lower chamber; wherein both the upper chamber and the lower chamber comprise a medium for culturing hepatocytes; (ii) a permeable membrane separating the upper chamber and the lower chamber; and (iii) a layer of hepatocyte-like cells grown on the permeable membrane, wherein the hepatocyte-like cells are differentiated from a population of pluripotent stem cells having a modified ABCB11 gene. In such an in vitro cell culture system, the hepatocyte-like cells are generated by any of the methods disclosed herein.

In still another aspect, the present disclosure provides a method for identifying an agent for treating a cholestatic liver disease, the method comprising: (i) providing an in vitro cell culture system as disclosed herein, (ii) adding a bile acid to the lower chamber, (iii) culturing the hepatocyte-like cells in the presence of a candidate agent; (iv) measuring the concentration of the bile acid in the upper chamber and/or in the lower chamber; and (v) identifying the candidate agent as an agent for treating a cholestatic liver disease, if the candidate agent changes the bile acid concentration determined in step (iv) as compared with the in vitro cell culture system in the absence of the candidate agent.

Further, the present disclosure provides a method for identifying an agent which disrupts bile acid transport and/or synthesis, the method comprising: (i) providing an in vitro cell culture system; (ii) adding a bile acid to the lower chamber; (iii) culturing the hepatocyte-like cells in the presence of a candidate agent; (iv) measuring the concentration of the bile acid in the upper chamber and/or in the lower chamber; and (v) identifying the candidate agent as an agent which disrupts bile acid transport and/or synthesis, if the candidate agent changes the bile acid concentration determined in step (iv) as compared with the in vitro cell culture system in the absence of the candidate agent. In such an in vitro cell culture system the hepatocyte-like cells are generated by any of the methods disclosed herein and have a functional apico-basolateral polarity, transport of bile acids and/or de novo synthesis of bile acids prior to the addition of the candidate agent.

Other features or advantages of the present invention will be apparent from the following drawings and detailed description of several examples, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A-1D include diagrams showing the generation of BSEP/ABCB11^{R1090X} mutant human iPSCs. FIG. 1A: a diagram of the gene map of BSEP/ABCB11 and location of R1090X, truncating mutation. FIG. 1B: a diagram showing the CRISPR/Cas9 genome editing was designed to replace the codon of CGA (arginine) with TGA (stop codon). FIG. 1C: a gel showing restriction enzyme digestion with BspHI identified correctly targeted clones of iPSCs (SEQ ID NO:1 and SEQ ID NO:2). FIG. 1D: microscopic bright field images of iPSCs. The cloned iPSCs with BSEP-R1090X mutations (BSEP^{R1090X}) showed comparable morphology to the parental iPSC colonies. (Scale bar: 100 μm)

FIGS. 2A-2E include graphs and images showing hepatic differentiation of BSEP^{R1090X} iPSCs and BSEP protein expression. FIG. 2A: bar graphs showing albumin concentration (A Left) of the culture supernatant in the upper and lower chambers measured with ELISA. The supernatant was collected 24 hours after medium changes. (A center) At the final stage of hepatic differentiation, i-Hep were dissociated with Trypsin and total cell counts were determined. (ns=not significant, *=p<0.05, n=5 or more). (A right) Albumin secretion per i-Hep cell at the final stage of hepatic differentiation. Normal and BSEP^{R1090X} hepatocytes (i-Hep) exhibited comparable albumin secretion into the culture medium. FIG. 2B: conventional light microscopic images of Hematoxylin and Eosin staining of normal and BSEP^{R1090X} i-Hep. Scale bar: 50 uM. FIG. 2C: immunofluorescent staining of normal and BSEP^{R1090X} i-Hep at the final stage of the differentiation protocol. Hepatocyte markers, HNF4a and CPS1, were detected both in normal and BSEP^{R1090X}. An endoderm marker of E-cadherin was detected on cell membrane. A tight junction protein, ZO1, was located at borders of cells. Nuclei were stained with Hoechst. (Scale bar: 10 μm) FIG. 2D: a western blotting to detect proteins of normal BSEP and truncated BSEPR1090X from cell lysates of i-Hep. BSEP^{R1090X} i-Hep showed a faint band at the lower level compared to the normal i-Hep lysate. Na—K ATPase (ATP1A1) was included as a loading control. FIG. 2E: immunofluorescent image of liver tissue in paraffin sections from a healthy subject and the patient with BSEP^{R1090X} truncating mutation. BSEP is localized at the canalicular membrane structure in the hepatocytes of a healthy subject. The protein with BSEP^{R1090X} mutation is localized in the cytosol, with a clustering pattern, in the hepatocytes of the patient with PFIC2. (Scale bar: 10 μm)

FIGS. 3A-3B include electron microscopic images showing the cellular ultrastructure of BSEP^{R1090X} i-Hep recapitulates the abnormalities observed in the liver tissue of the patient with PFIC2. FIG. 3A: electron microscopic images of normal (left column) and BSEP^{R1090X} i-Hep (right column). Cells on the Transwell membrane were cross-sectioned. Normal i-Hep showed dense microvilli on the apical surface whereas BSEP^{R1090X} i-Hep showed sparse microvilli (black arrows). Basolateral membrane irregularity with wider interstitial space between hepatocytes was observed in BSEP^{R1090X} (white arrowheads). FIG. 3B: electron microscopic images of liver tissues from a healthy subject (left column) and the patient with PFIC2 (right column). The hepatocytes of the patient's liver showed decreased microvilli in the bile canaliculus (black arrows) and wider interstitial space between basolateral membranes of adjacent cells (white arrowheads). (Scale bar: 2 μm).

FIGS. 4A-4H include graphs and images showing the basolateral-to-apical transport of TCA in BSEP^{R1090X} i Hep. FIG. 4A: a diagram showing the experimental schemes of exogenous TCA transport from the lower chamber to the upper chamber. FIG. 4B: a graph showing the amount of bile acid in the upper chamber was measured at 24 h and 48 h after loading TCA in the lower chamber. (*p<0.05, n=5). FIG. 4C: a graph showing the percentage fraction of the sum of bile acids measured from the upper and lower chamber in a well at 0, 24, 48 hours after loading of TCA. Grey: Percentage fraction of bile acids measured in the lower chamber. Black: in the upper chamber. FIG. 4D: a diagram showing the experimental schemes of TCA transport from the upper chamber to the lower chamber. FIG. 4E: a graph showing the mass of bile acid in culture medium in the lower chamber, 24 h and 48 h after loading TCA in the upper chamber. (*p<0.05, n=5) FIG. 4F: a graph showing the percentage fraction of measured bile acid in a well at 0, 24, 48 hours after loading of TCA. Grey: Percentage fraction of bile acids measured in the lower chamber. Black: in the upper chamber. FIG. 4G: a table showing the permeability of the monolayer between the upper and lower chamber measured with dextrose conjugated fluorescent probe (10,000 MW Alexa fluor). The probe was measured in the culture supernatant in the chambers 48 hours after loading into the opposite chambers; described as percentage (±SD) of the initial amount of loaded probe. FIG. 4H: a graph showing the monolayer barrier function measured with trans-epithelial electrical resistance (TEER) between upper and lower chamber via monolayer and transwell membrane. (ns: not significant, *: p<0.05, n=5 or more).

FIGS. 5A-5B include diagrams and graphs showing the intrahepatic accumulation of D4-TCA in BSEP^{R1090X} i-Hep during transcellular transport FIG. 5A: a diagram and graph showing the transport assay of isotope labelled TCA (D4-TCA) to determine intracellular accumulation of TCA over a 24 hour-period. D4-TCA (1 μM) was added into the lower chamber. The amount of TCA was quantified by mass spectrometry in the cell lysates collected at 4, 12, and 24 hours after loading. The amount of D4-TCA is calculated per well. (* p<0.05, n=4). FIG. 5B: a diagram and graph showing the uptake assay of D4-TCA. D4-TCA (10 μM) was added into the lower chamber and cell lysates were collected after 5 min and 15 min incubation with or without sodium in the culture medium. Without sodium in culture medium, D4-TCA was not taken up by the i-Hep. (*: p<0.05, n=3).

FIGS. 6A-6C include a diagram and graphs showing BSEP^{R1090X} i-Hep exports intracellular TCA back into the lower chambers via basolateral MRP4. FIG. 6A: a diagram and graphs showing the wash-out assay to determine the transport (efflux) direction of intracellular D4-TCA. After 1 hour of D4-TCA incubation in the lower chamber (1004), i-Hep cells were washed with medium and placed in a fresh medium. The intracellular D4-TCA was exported into the fresh medium in the upper and lower chambers and measured at 5, 15, 30 and 60 minutes by mass spectrometry. BSEP^{R1090X} i-Hep showed basolateral excretion of TCA as opposed to normal i-Hep which excretes TCA apically. (*: p<0.05, n=5 or more). FIG. 6B: a graph showing the gene expressions of hepatic ABC transporters in i-Hep cells at the final stage of differentiation were measured by quantitative real-time PCR (n=4). After normalized to 18S rRNA, each gene expression level was shown relative to the expression level in normal i-Hep. When compared to normal i-Hep (*p<0.05), the BSEP^{R1090X} i-Hep expressed more ABCC4/MRP4. 18S rRNA housekeeping gene expression did not differ between cell types (p>0.05). FIG. 6C: a graph showing the wash-out assay to determine the role of MRP4 in intracellular-to-basolateral export of D4-TCA by using MRP4 inhibitor (Ceefourin1). After 1 hour of D4-TCA incubation in the lower chamber (10 μM), i-Hep cells were washed and placed in a fresh medium with or without MRP4 inhibitor. The exported D4-TCA in the lower chamber was measured by mass spectrometry at 5, 15, and 30 minutes. At 15 and 30 min, MRP4 inhibitor decreased D4-TCA export towards lower chamber (* p<0.05, n=4 or more).

FIGS. 7A-7I include diagrams and graphs showing that maturing BSEP^{R1090X} i-Hep adapt export synthesized bile acids via the basolateral membrane and respond to exogenous bile acids FIG. 7A: shows the gene expression of CYP7a in i-Hep is measured by RT-PCR at the last stages of differentiation. In both normal and BSEP^{R1090X} i-Hep, CYP7a expression increased from Day 17 of culture to Day 21. The fold change of gene expression was based to the values of day 17. (*p<0.05: Day 17 vs Day 21, n=3) FIG. 7B: shows the amount of endogenous taurocholic acid (TCA) exported into the upper chamber (black) and lower chamber (grey) was measured by mass spectrometry. After the incubation in fresh culture medium for 48 hours, the TCA concentration in the culture supernatant from the upper and lower chambers was determined. Normal i-Hep exported endogenous TCA towards the upper chamber (apical domain) whereas BSEP^{R1090X} i-Hep towards the lower chamber (basolateral domain) Total amount of TCA synthesized by BSEP^{R1090X} i-Hep was less than normal i-Hep. (*=p<0.05, black: lower chamber normal vs BSEP^{R1090X}, blue: upper chamber normal vs BSEP^{R1090X}, purple: upper chamber vs lower chamber of each i-Hep) FIG. 7C: shows the amount of intracellular TCA was measured from cell lysates after 48 hours incubation. Intracellular TCA in normal and BSEP^{R1090X} i-Hep were comparable. FIG. 7D: shows a schematic description of experiments design in normal i-Hep. Labelled TCA, D4-TCA, was added to the lower chamber. After the incubation, TCA (endogenous and D4-TCA) in the culture medium was measured separately. FIG. 7E: shows the amount of endogenous TCA secreted into the upper and lower chambers was measured in the conditions cultured with or without exogenous D4-TCA. The exogenous D4-TCA suppressed endogenous synthesis of TCA. (*=p<0.05, black: lower chambers cultured with vs without D4-TCA, blue: upper chambers cultured with vs without D4-TCA, purple: upper chamber vs lower chamber of each i-Hep) FIG. 7F: shows a schematic description of experiments design in BSEP^{R1090X} i-Hep. FIG. 7G: shows the amount of endogenous TCA secreted into the upper and lower chambers was measured in the conditions cultured with or without exogenous D4-TCA. FIG. 7H: shows the intracellular TCA, endogenous and D4-TCA, measured separately from the cell lysate after the incubation. Exogenous D4-TCA accumulated in normal and BSEP^{R1090X} i-Hep is comparable. (ns: p>0.05). FIG. 7I: shows the gene expression of the FXR pathway was determined by RT-PCR. In both normal and BSEP^{R1090X} i-Hep, CYP7a was down-regulated and SHP was up-regulated when D4-TCA was added into the lower chamber for 12 h and 24 h. No significant change was found in FXR expression. The fold change of gene expression was based to the values in the condition cultured without D4-TCA. (*p<0.05, n=3 or more)

FIGS. 8A-8B include a model representing mechanism regulating de novo bile acid synthesis in BSEP deficient hepatocytes FIG. 8A: a diagram showing in normal hepatocytes, synthesized bile acids are exported to the bile canaliculus and return to the sinusoid by the hepato-enteric circulation (1). The bile acids in the sinusoid are taken up by hepatocytes and suppress de novo synthesis mediated by the intracellular concentration of bile acids (2 and 3). FIG. 8B: a diagram showing in BSEP deficient hepatocytes, synthesized bile acids are exported to the sinusoid and accumulate in the systemic circulation (1). When taken up from the sinusoid, the intracellular bile acids suppress de novo bile acid synthesis while being exported to the sinusoid via the basolateral membrane (2 and 3).

### DETAILED DESCRIPTION OF THE INVENTION

Genetic defects affecting bile acid transport pathways present in several clinical phenotypes including Progressive Familial Intrahepatic Cholestasi (PFIC), Benign Recurrent Intrahepatic Cholestasis (BRIC), and Intrahepatic Cholestasis of Pregnancy (ICP). Progressive familial intrahepatic cholestasis (PFIC) is a class of chronic cholestasis disorders that begin in infancy and usually progress to cirrhosis within the first decade of life. The average age at onset is 3 months, although some patients do not develop jaundice until later, even as late as adolescence. PFIC can progress rapidly and cause cirrhosis during infancy or may progress relatively slowly with minimal scarring well into adolescence. Few patients have survived into the third decade of life without treatment.

PFIC types 1 and 2 are rare, but the exact frequency is unknown. Incidence is estimated at 1:50,000 to 1:100,000 births. All forms of progressive familial intrahepatic cholestasis are lethal in childhood unless treated. Morbidity is the result of chronic cholestasis. Pruritus is more pronounced in PFIC types 1 and 2 and often occurs out of proportion to the level of jaundice, which is often low grade and can wax and wane. The pruritus may be disabling and usually does not respond to medical therapy. Greater understanding of individualized pathways driving disease-causing pathologies and response to therapy, and the clinical translation of these data, is needed to design personalized management strategies at an early stage of the disease.

The present disclosure is based, at least in part, in the development of an in vitro disease model for BSEP deficiency, which can be used to improve understanding of genetic cholestatic liver disease and identify a candidate agent for treating the disease. In some embodiments, the in vitro disease model disclosed herein involves gene editing in isogenic iPSCs through CRISPR/Cas9 technology. Such an in vitro model can be used to elucidate a direct molecular consequence of a single nucleotide variant found in patients. This system allows for direct determination of the cellular and biochemical effects of previously unreported genetic variants and determination of the molecular consequence of missense mutations, often reported as “variant of unknown clinical significance”. As the knowledge of disease-causing variants further accumulates, it would be relied on to predict the clinical course from the genotype and design personalized management strategies at an early stage of the disease. In another aspect, the in vitro model as disclosed herein can be used to identify whether a candidate agent will disrupt bile acid transport and/or synthesis in unmodified hepatocyte-like cells (e.g., hepatocyte like-cells produced from wild-type PS cells). This system allows for determination that a candidate agent produces or does not produce side effects related to bile acid metabolism and/or transport.

### I. Methods of Producing Hepatocyte-Like Cells In Vitro

Aspects described herein stem from, at least in part, development of methods that efficiently direct differentiation of pluripotent stem (PS) cells into hepatocyte-like cells. In particular, the present disclosure provides, inter alia, an in vitro culturing process for producing a population of hepatocyte-like cells from pluripotent stem cells and the resultant hepatocyte-like cells show a functional apico-basolateral polarity, including canalicular function, specifically in bile acid transport and bile acid de novo synthesis, from unmodified pluripotent stem cells (e.g., from a human subject). In some embodiments, this culturing process may involve multiple differentiation stages (e.g., 2, 3, or more). Alternatively, or in addition, the culturing process may involve culture of the cells on a permeable membrane which separates and upper and lower chamber in a cell culture vessel. In some embodiment, the total time period for the in vitro culturing process described herein can range from about 17-27 days (e.g., 20-26 days, 20-23 days, or 19-23 days). In one example, the total time period is about 22 days.

In some embodiments, the methods for producing hepatocyte-like cells as disclosed herein may include multiple differentiation stages (e.g., 2, 3, 4, or more). For example, a endoderm differentiation step, e.g., the culturing of the hPS cells under differentiation conditions to obtain cells of the definitive endoderm (DE cells), a hepatic specification step, e.g., the culturing of the obtained DE cells under differentiation conditions to obtain the hepatic progenitor cells, and a hepatic maturation step, e.g., culturing the hepatic progenitor cells under conditions to obtain hepatocyte-like cells.

Existing methods for producing human hepatocytes often fail to form functional apico-basolateral polarity. Thus, there is a lack of a suitable experimental system for dynamic tracing of transcellular transport of bile acids. The in vitro model described herein can provide a reliable source of hepatocyte-like cells with transcellular transport and de novo synthesis of bile acids. The pluripotent stem (PS) cell-derived hepatocyte-like cells can be used in various applications, including, e.g., but not limited to, as an in vitro model for modeling genetic cholestatic liver diseases or disorders, drug discovery and/or developments.

Accordingly, embodiments of various aspects described herein relate to methods for generation of hepatocyte-like cells from PS cells, cells produced by the same, and methods of use.

A. Pluripotent Stem Cells

In some embodiments, the in vitro culturing system disclosed herein may use pluripotent stem cells (e.g., human pluripotent stem cells) as the starting material for producing hepatocyte-like cells. As used herein, “pluripotent” or “pluripotency” refers to the potential to form all types of specialized cells of the three germ layers (endoderm, mesoderm, and ectoderm); and is to be distinguished from “totipotent” or “totipotency”, that is the ability to form a complete embryo capable of giving rise to offsprings. As used herein, “human pluripotent stem cells” (hPSC) refers to human cells that have the capacity, under appropriate conditions, to self-renew as well as the ability to form any type of specialized cells of the three germ layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson et al. (1998), Heins et. al. (2004), as well as induced pluripotent stem cells [see, e.g. Takahashi et al., (2007); Zhou et al. (2009); Yu and Thomson in Essentials of Stem Cell Biology (2nd Edition]. The various methods described herein may utilize hPS cells from a variety of sources. For example, hPS cells suitable for use may have been obtained from developing embryos by use of a nondestructive technique such as by employing the single blastomere removal technique described in e.g. Chung et al (2008), further described by Mercader et al. in Essential Stem Cell Methods (First Edition, 2009). Additionally or alternatively, suitable hPS cells may be obtained from established cell lines or may be adult stem cells.

In some aspects, the pluripotent stem cells for use according to the disclosure may be human embryonic stem cells (hESs). Various techniques for obtaining hES cells are known to those skilled in the art. In some instances, the hES cells for use according to the present disclosure are ones, which have been derived (or obtained) without destruction of the human embryo, such as by employing the single blastomere removal technique known in the art. See, e.g., Chung et al., Cell Stem Cell, 2(2):113-117 (2008), Mercader et al., Essential Stem Cell Methods (First Edition, 2009). Suitable hES cell lines can also be used in the methods disclosed herein. Examples include, but are not limited to, cell lines SA167, SA181, SA461 (Cellartis AB, Goteborg, Sweden) which are listed in the NIH stem cell registry, the UK Stem Cell bank and the European hESC registry and are available on request. Other suitable cell lines for use include those established by Klimanskaya et al., Nature 444:481-485 (2006), such as cell lines MA01 and MA09, and Chung et al., Cell Stem Cell, 2(2):113-117 (2008), such as cell lines MA126, MA127, MA128 and MA129, which all are listed with the International Stem Cell Registry (assigned to Advanced Cell Technology, Inc. Worcester, Mass., USA).

Alternatively, the pluripotent stem cells for use in the methods disclosed herein may be induced pluripotent stem cells (iPSCs) such as human iPSCs. As used herein “hiPS cells” refers to human induced pluripotent stem cells. hiPS cells are a type of pluripotent stem cells derived from non-pluripotent cells—typically adult somatic cells—by induction of the expression of genes associated with pluripotency, such as SSEA-3, SSEA-4,TRA-1-60,TRA-1-81,Oct-4, Sox2, Nanog and Lin28. Various techniques for obtaining such iPSC cells have been established and all can be used in the present disclosure. See, e.g., Takahashi et al., Cell 131(5):861-872 (2007); Zhou et al., Cell Stem Cell. 4(5):381-384 (2009); Yu and Thomson in Essentials of Stem Cell Biology (2nd Edition, Chapter 4)]. It is also envisaged that the endodermal and/or hepatic progenitor cells may also be derived from other pluripotent stem cells such as adult stem cells, cancer stem cells or from other embryonic, fetal, juvenile or adult sources.

B. Genetic Modification of Pluripotent Stem Cells

In some embodiments, the pluripotent stem cells used in the in vitro culturing system disclosed herein for producing hepatocyte-like cells may be genetically modified such that the ABCB11 gene, which encodes a Bile Salt Export Pump (BSEP) protein, is disrupted. As used herein, the term “BSEP” is intended to mean the bile transporter bile salt export pump. Accordingly, the present disclosure also provides methods of preparing such genetically modified pluripotent stem cells. As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional or has substantially reduced activity. In some embodiments, a disrupted gene does not express (e.g., encode) a functional protein.

The ABCB11/BSEP protein contains 12 transmembrane domains and 2 intracellular nucleotide-binding domains. In some embodiments, the targeted modification of ABCB11/BSEP is at the R1090 position, located in exon 25. In a specific example, the modification results in a truncation at R1090 which induces a BSEP protein without a functional C-terminal domain, lacking the second nucleotide-binding domain of Walker A and B and a conserved signature C motif of ATP-binding cassette (ABC). The resulting peptide is a short BSEP with an unpaired, single, intracellular ABC domain. The instant disclosure demonstrates that truncated versions of BSEP, such as the R1090X mutant, exhibits dysfunction in hepatocyte-like cells.

In another exemplary embodiment, the targeted modification results in a truncating mutation, R1057X. The R1057X truncating mutation was studied in a transfection model in MDCK II cells and showed stable expression level but low transport activity. Kagawa et al., American Journal of Physiology Gastrointestinal and Liver Physiology 294:G58-6 (2008).

Alternatively, the genetically modified pluripotent stem cells may have a disrupted gene involved in a bile acid transport or synthesis pathway in hepatocytes, for example, a gene know or thought to be involved in a genetic cholestatic liver disease (e.g., Progressive Familial Intrahepatic Cholestasis (PFIC), Benign Recurrent Intrahepatic Cholestasis (BRIC), and Intrahepatic Cholestasis of Pregnancy (ICP)). Non-limiting examples of gene contributors of PFIC, BRIC, and/or ICP include ATP8B1/FIC1 (gene on chromosome 18q21-22), and ABCB4/MDR3 (gene on chromosome 7q21). As used herein, the term “MDR” is intended to mean multi-drug resistance transporter. MDR 1 and 3 are members of the ATP-binding cassette (ABC) family of transporters. MDR 1 is important in regulating the traffic of drugs, peptides and xenobiotics into the body and in protecting the body against xenobiotic insults and drug toxicity, while MDR 3 is essential for phospholipid secretion into bile.

Techniques such as CRISPR (particularly using Cas9 and guide RNA), editing with zinc finger nucleases (ZFNs) and transcription activator-like effector nucleases (TALENs) may be used to produce the genetically engineered pluripotent stem cells.

‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell. Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted modification can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

In an exemplary embodiment, the CRISPR/Cas9 gene editing technology is used for producing the genetically engineered pluripotent stem cells. Typically, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. Any known CRISPR/Cas9 methods can be used in the methods disclosed herein. See also Examples below.

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the Fold nuclease with a zinc finger DNA binding domain

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the Fold nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the gene editing nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

C. Endoderm Differentiation

The in vitro culturing system disclosed herein may involve a step of endoderm differentiation to differentiate any of the PSCs disclosed herein to definitive endoderm. Suitable conditions for endoderm differentiation are known in the art (see, e.g., Hay 2008, Brolen 2010 and Duan 2010, and WO 2009/013254 A1) and/or disclosed in Examples below. As used herein “definitive endoderm (DE)” and “definitive endoderm cells (DE cells)” refers to cells exhibiting protein and/or gene expression as well as morphology typical to cells of the definitive endoderm or a composition comprising a significant number of cells resembling the cells of the definitive endoderm. The definitive endoderm is the germ cell layer which gives rise to cells of the intestine, pancreas, liver and lung. DE cells may generally be characterized, and thus identified, by a positive gene and protein expression of the endodermal markers FOXA2, CXCR4, HHEX, SOX17, GATA4 and GATA6. The two markers SOX17 and CXCR4 are specific for DE and not detected in hPSC, hepatic progenitor cells or hepatocytes. Lastly, DE cells do not exhibit gene and protein expression of the undifferentiated cell markers Oct4, SSEA-3, SSEA-4, TRA-1-60, TRA-1-81, but can show low Nanog expression.

Generally, in order to obtain DE cells, PSCs such as hPSC cells can be cultured in an endoderm differentiation medium comprising activin, such as activin A or B. The endoderm differentiation medium may further include a histone deacetylase (HDAC) inhibitor, such as Sodium Butyrate (NaB), Phenylbutyrate (PB), valproate, trichostatin A, Entinostat or Panobinstat. The endoderm differentiation medium may optionally further comprise one or more growth factors, such as FGF1, FGF2 and FGF4, and/or serum, such as FBS or FCS or a serum replacement such as B27+insulin. The endoderm differentiation medium may comprise a GSK3-inhibitor, such as, e.g., CHIR99021, or an activator of Wnt signaling, such as Wnt3A. The endoderm differentiation medium may further include a Rho-associated protein kinase (ROCK) inhibitor. Non-limiting examples of Rho-associated protein kinase (ROCK) inhibitors include, but are not limited to, Y27632, HA-100, H-1152, (+)-trans-4-(1-aminoethyl)-1-(pyridin-4-ylaminocarbony I) cyclohexane dihydro-chloride monohydrate (described in WO0007835 & WO00057913), imidazopyridine derivatives (described in U.S. Pat. No. 7,348,339), substituted pyrimidine and pyridine derivatives (described in U.S. Pat. No. 6,943,172) and substituted isoquinoline-sulfonyl compounds (described in EP00187371), or GSK429286A, or Thiazovivin, or an analog or derivative thereof.

The concentration of activin is usually in the range of about 50 to about 200 ng/ml, such as about 80 to about 120 ng/ml. Activin may, for example, be present in the endoderm differentiation medium at a concentration of about 90 ng/ml or about 100 ng/ml. As used herein, the term “Activin” is intended to mean a TGF-beta family member that exhibits a wide range of biological activities including regulation of cellular proliferation and differentiation such as “Activin A” or “Activin B”. Activin belongs to the common TGF-beta superfamiliy of ligands.

The concentration of the HDAC inhibitor is usually in the range of about 0.1 to about 1 mM. The HDAC inhibitor may, for example, be present in the endoderm differentiation medium at a concentration of about 0.4 mM or about 0.5 mM. In one aspect, the HDAC inhibitor is removed from the endoderm differentiation medium after about 3 days. In another aspect, the HDAC inhibitor is added on day 2 and removed on day 5 of culturing PSCs in an endoderm differentiation medium. As used herein HDAC inhibitors refers to Histone deacetylase inhibitors, such as Sodium Butyrate (“NaB”), Phenyl Butyrate (“PB”), Trichostatin A and Valproic Acid (“VA”).

The concentration of serum, if present, is usually in the range of about 0.1 to about 2% v/v, such as about 0.1 to about 0.5%, about 0.2 to about 1.5% v/v, about 0.2 to about 1% v/v, about 0.5 to 1% v/v or about 0.5 to about 1.5% v/v. Serum may, for example, if present, in the endoderm differentiation medium may be at a concentration of about 0.2% v/v, about 0.5% v/v or about 1% v/v. In one aspect, the endoderm differentiation medium omits serum and instead comprises a suitable serum replacement such as B27+insulin.

The concentration of the activator of Wnt signaling is usually in the range of about 0.05 to about 90 ng/ml, such as about 50 ng/ml. As used herein, “activator of Wnt signaling” refers to a compound which activates Wnt signaling. The concentration of the GSK3 inhibitor, if present, is usually in the range of about 0.1 to about 10 μM, such as about 0.05 to about 5 μM. The concentration of the ROCK inhibitor, if present, is typically in the range of 1 μM to about 20 such as 10 μM.

The culture medium forming the basis for the endoderm differentiation medium may be any culture medium suitable for culturing PS cells such as RPMI 1640 or advanced medium, Dulbecco's Modified Eagle Medium (DMEM), HCM medium, HBM medium or Williams E based medium. Thus, the differentiation medium may be RPMI 1640 or advanced medium comprising or supplemented with the above-mentioned components. Alternatively, the differentiation medium may be DMEM comprising or supplemented with the above-mentioned components. The endoderm differentiation medium may thus also be HCM medium comprising or supplemented with the above-mentioned components. The endoderm differentiation medium may thus also be HBM medium comprising or supplemented with the above-mentioned components. The endoderm differentiation medium may thus also be Williams E based medium comprising or supplemented with the above-mentioned components. In one embodiment, the endoderm differentiation medium comprises RPMI1640 containing, in a range of about 1-3%, B27 serum replacement (ThermoFisher).

In some embodiments, the endoderm differentiation medium comprises, consists essentially of, or consists of, an activin, an inhibitor of class I histone deacetylase and an activator of Wnt signaling pathway or GSK3 inhibitor. In other embodiments, the endoderm differentiation medium comprises, consists essentially of, or consists of, an activin, an activator of Wnt signaling pathway or GSK3 inhibitor and a ROCK inhibitor. In another embodiment, the endoderm differentiation medium comprises, consists essentially of, or consists of 1 mM sodium butyrate, Wnt3a 50 ng/mL and Activin A 100 ng/mL, wherein when ‘consisting of’ the medium includes RPMI and a suitable serum replacement (e.g., B27+insulin). In yet another embodiment, the endoderm differentiation medium comprises, consists essentially of, or consists of, Wnt3a 50 ng/mL, Activin A 100 ng/mL, 10 μM Y 27632, wherein when ‘consisting of’ the medium includes RPMI and a suitable serum replacement (e.g., B27+insulin). In still yet another embodiment, the endoderm differentiation medium comprises, consists essentially of, or consists of, 3 μM CHIR99021, 100 ng/mL Activin A, 1 mM sodium butyrate, wherein when ‘consisting of’ the medium includes RPMI and a suitable serum replacement (e.g., B27+insulin). In another embodiment, the endoderm differentiation medium comprises, consists essentially of, or consists of, 3 μM CHIR99021, 100 ng/mL Activin A, 10 μM Y 27632, wherein when ‘consisting of’ the medium includes RPMI and a suitable serum replacement (e.g., B27+insulin).

The PS cells are normally cultured for up to 6 days in suitable endoderm differentiation medium in order to obtain hepatic progenitor cells. For example, the PS cells may be cultured in suitable differentiation medium for about 4 to about 14 days, such as for about 5 to 8 days. In some embodiments, the PS cells are cultured in a cell culture vessel coated with at least one extracellular matrix protein (e.g., laminin or Matrigel) during contact with the endoderm differentiation medium. In some embodiments, the PS cells are dissociated after about 5 days and placed on a permeable membrane, optionally coated with at least one extracellular matrix protein, in a cell culture vessel with an upper and lower chamber separated by the permeable membrane. The PS cells are then contacted with endoderm differentiation medium for the remaining time to induce DE cells, such as about 1-2 days. The PS cells may be dissociated and collected in suspension (e.g., through contact with TrypLE) and then placed in the cell culture vessel having an upper chamber and a lower chamber separated by a permeable membrane. Suitable cell culture vessels are not particularly limited and can include any vessel or insert added thereto where the upper and lower chambers are separated by a permeable membrane. Suitable examples of permeable membranes include but are not limited to polycarbonate, polyester (PET), and collagen-coated polytetrafluoroethylene (PTFE).

In some examples, the method disclosed herein may be performed by culturing the population of pluripotent stem cells in the endoderm differentiation medium for about 5-8 days. In one specific example, endoderm differentiation can be performed by (a) culturing the population of pluripotent stem cells in a first endoderm differentiation medium for one day, wherein the first endoderm differentiation medium comprises an activin, insulin, the activator of Wnt signaling pathway, and the ROCK inhibitor; (b) culturing the population of pluripotent stem cells in a second endoderm differentiation medium following step (a) for one day, wherein the second endoderm differentiation medium comprises an activin, insulin, the activator of Wnt signaling pathway, and the inhibitor of class I histone deacetylase; (c) culturing the population of pluripotent stem cells in a third endoderm differentiation medium following step (c) for two days, wherein the third endoderm differentiation medium comprises an activin, insulin, the GSK3 inhibitor, and the inhibitor of class I histone deacetylase; (d) culturing the population of pluripotent stem cells in a fourth endoderm differentiation medium following step (c) for one day, wherein the fourth endoderm differentiation medium comprises an activin, insulin, the GSK3 inhibitor, and the ROCK inhibitor; and (e) culturing the population of pluripotent stem cells in a fifth endoderm differentiation medium following step (d) for one day, wherein the fifth endoderm differentiation medium comprises an activin, insulin, and the GSK3 inhibitor. In some instances, after step (c) and prior to step (d), the population of pluripotent stem cells can be placed on a permeable membrane.

D. Hepatic Specification

Following the endoderm differentiation step, the obtained DE cells can be further cultured in a hepatic specification medium to obtain hepatic progenitor cells. As used herein, “hepatic progenitors” or “hepatic progenitor cells” refers to cells which have entered the hepatic cell path and give rise to hepatocyte. “Hepatic progenitors” are thus distinguished from “endodermal cells” in that they have lost the potential to develop into cells of the intestine, pancreas and lung. “Hepatic progenitors” may generally be characterized, and thus identified, by a positive gene and protein expression of the early hepatic markers EpCAM, c-Met (HGF-receptor), AFP, CK19, HNF6, C/EBPa and β. They do not exhibit gene and protein expression of the DE-markers CXCR4 and SOX17. Lastly, “hepatic progenitors” do not exhibit gene and protein expression of the undifferentiated cell markers Oct4, SSEA-3, SSEA-4, TRA-1-60 and TRA-1-81 nor the mature hepatic markers CYP1A2, CYP2C9, CYP19, CYP3A4, CYP2B6 and PXR.

In general, in order to obtain hepatic progenitor cells, DE cells are cultured in a hepatic differentiation medium comprising one or more growth factors, such as a fibroblast growth factor (FGF) (e.g., FGF1, FGF2 and FGF4), and one or more bone morphogenic proteins (BMP), such as BMP2 and BMP4. As used herein, the term “FGF” means fibroblast growth factor, preferably of human and/or recombinant origin, and subtypes belonging thereto are e.g. “bFGF” (means basic fibroblast growth factor, sometimes also referred to as FGF2) and FGF4. “aFGF” means acidic fibroblast growth factor (sometimes also referred to as FGF1). As used herein, the term “BMP” means Bone Morphogenic Protein, preferably of human and/or recombinant origin, and subtypes belonging thereto are e.g. BMP4 and BMP2.

The concentration of the one or more growth factors may vary depending on the particular compound used. The concentration of FGF2, for example, is usually in the range of about 2 to about 50 ng/ml, such as about 2 to about 20 ng/ml. FGF2 may, for example, be present in the specification medium at a concentration of 9 or 10 ng/ml. The concentration of FGF1, for example, is usually in the range of about 50 to about 200 ng/ml, such as about 80 to about 120 ng/ml. FGF1 may, for example, be present in the specification medium at a concentration of about 100 ng/ml. The concentration of FGF4, for example, is usually in the range of about 20 to about 40 ng/ml. FGF4 may, for example, be present in the specification medium at a concentration of about 30 ng/ml. The concentration of the one or more BMPs, is usually in the range of about 50 to about 300 ng/ml, such as about 50 to about 250 ng/ml, about 100 to about 250 ng/ml, about 150 to about 250 ng/ml, about 50 to about 200 ng/ml, about 100 to about 200 ng/ml or about 150 to about 200 ng/ml. The concentration of BMP2, for example, is usually in the range of about 2 to about 50 ng/ml, such as about 10 to about 30 ng/ml. BMP2 may, for example, be present in the hepatic specification medium at a concentration of about 20 ng/ml.

The culture medium forming the basis for the hepatic specification medium may be any culture medium suitable for culturing human endodermal cells such as RPMI 1640 or advanced medium, Dulbecco's Modified Eagle Medium (DMEM), HCM medium, HBM medium or Williams E based medium. Thus, the hepatic specification medium may be RPMI 1640 or advanced medium comprising or supplemented with the above-mentioned components. Alternatively, the hepatic specification medium may be DMEM comprising or supplemented with the above-mentioned components. The hepatic specification medium may thus also be HCM medium comprising or supplemented with the above-mentioned components. The hepatic specification medium may thus also be HBM medium comprising or supplemented with the above-mentioned components. The hepatic specification medium may thus also be Williams E based medium comprising or supplemented with the above-mentioned components. In some embodiments, the DE cells are cultured in a cell culture vessel coated with at least one extracellular matrix protein (e.g., laminin) during contact with the hepatic specification medium.

In other embodiments, the hepatic specification medium comprises, consists essentially of, or consists of, bFGF and BMP4. In another embodiment, the endoderm differentiation medium comprises, consists essentially of, or consists of 50 ng/ml bFGF and 20 ng/ml BMP4, wherein when ‘consisting of’ the medium includes RPMI and a suitable serum replacement (e.g., B27+insulin).

For specification into hepatic progenitor cells, DE cells are normally cultured for up to 3 days in differentiation medium as described above. The DE cells may, for example, be cultured in differentiation medium for about 2 to about 4 days. In some embodiments, the DE cells are maintained in the cell culture vessel comprising an upper and lower chamber separated by a permeable membrane, optionally coated with at least one extracellular matrix protein, during specification to hepatic progenitor cells, wherein the DE cells are in contact with the permeable membrane.

E. Hepatocyte Maturation

The hepatocyte progenitor cells obtained from the hepatocyte specification step may be further cultured in a hepatic maturation medium to obtain the hepatocyte-like cells. As used herein, “hepatocyte” or “hepatocyte-like cells” refers to fully differentiated hepatic cells. “Hepatocytes” or “hepatocytes-like cells” may generally be described, and thus identified, by a positive gene and protein expression of the mature hepatic markers CYP1A2, CYP3A4, CYP2C9, CYP2C19, CYP2B6, GSTA1-1, OATP-2, NTCP, Albumin, PXR, CAR, and HNF4a (isoforms 1 +2) among others. Further, “hepatocytes” or “hepatocyte-like cells do not exhibit gene and protein expression of the undifferentiated cell markers Oct4, SSEA-3, SSEA-4, TRA-1-60 and TRA-1-81. Compared to DE cells, “hepatocytes” or “hepatocyte-like cells do not exhibit gene and protein expression of the DE cell markers SOX17 and CXCR4. Compared to “hepatic progenitors”, “hepatocytes” or “hepatocyte-like cells do not exhibit gene and protein expression of the hepatic progenitor markers Cytokeratin 19 and AFP. As meant herein, a gene or protein shall be interpreted as being “expressed”, if in an experiment measuring the expression level of said gene or protein, the determined expression level is higher than three times the standard deviation of the determination, wherein the expression level and the standard deviation are determined in 10 separate determinations of the expression level. The determination of the expression level in the 10 separate determinations is preferably corrected for background-signal. Moreover, the ‘hepatocyte-like cells’ is meant to include cells which have similar functionalities as primary hepatocytes, and in particular show phenotypical features of functional hepatocytes when exposed to bile acids. Said phenotypical features may include expression and polarization of bile acid transport proteins, uptake, transport, synthesis and/or excretion of bile acids at a level similar to primary hepatocytes. In particular, in the context of the present invention, hepatocyte-like cells are meant to include human embryonic stem cells differentiated into hepatocyte-like cells, human induced pluripotent stem cells differentiated into hepatocyte-like cells, or primary fibroblast transdifferentiated into hepatocyte-like cells.

In general, in order to obtain hepatocyte-like cells, hepatic progenitor cells are cultured in a hepatocyte maturation medium comprising one or more of a hepatocyte growth factor (HGF), one or more differentiation inducer (e.g., such as dimethylsulfoxide (DMSO), dexamethazone (DexM), omeprazole, Oncostatin M (OSM), rifampicin, desoxyphenobarbital, ethanol or isoniazide), transferrin, hydrocortisone and insulin, where the hepatocyte maturation medium preferably omits human epidermal growth factor (EGF). As used herein, the term “HGF” means Hepatocyte Growth Factor, preferably of human and/or recombinant origin. As used herein, the term “EGF” means Epidermal Growth Factor, preferably or human and/or recombinant origin.

The concentration of HGF, is usually in the range of about 5 to about 30 ng/ml. HGF may, for example, be present in the differentiation medium at a concentration of about 20 ng/ml. The concentration of DMSO, for example, is usually in the range of about 0.1 to about 2% v/v, such as about 0.1 to about 1.5% v/v, about 0.1 to about 1% v/v, about 0.25 to about 1% v/v, about 0.25 to about 0.75% v/v, about 0.5 to about 1.5% v/v, or about 0.5 to about 1% v/v. The concentration of OSM, for example, is usually in the range of about 1 to about 20 ng/ml, such as about 1 to about 15 ng/ml, about 5 to about 15 ng/ml, or about 7.5 to about 12.5 ng/ml. The concentration of DexM, for example, is usually in the range of about 0.05 to about 1 μM, such as about 0.05 to about 0.5 μM, about 0.05 to about 0.2 μM, about 0.05 to about 0.1 μM or about 0.1 to about 0.5 μM.

The hepatocyte maturation medium may further comprise serum, such as FBS or FCS. The concentration of serum, if present, is usually in the range of about 0.1 to about 5% v/v, such as about 0.1 to about 0.5%, 0.2 to 3% v/v, about 0.5 to about 2.5% v/v, about 0.5 to 1% v/v or about 1 to about 2.5% v/v. In some embodiments, the hepatocyte maturation medium further comprises one or more of BSA-fatty acid free (BSA-FAF), ascorbic acid, and GA-1000.

The culture medium forming the basis for the hepatocyte maturation medium may be any culture medium suitable for culturing human endodermal cells such as RPMI 1640 or advanced medium, Dulbecco's Modified Eagle Medium (DMEM), HCM medium, HBM medium or Williams E based medium. Thus, the hepatocyte maturation medium may be RPMI 1640 or advanced medium comprising or supplemented with the above-mentioned components. Alternatively, the hepatocyte maturation medium may be DMEM comprising or supplemented with the above-mentioned components. The hepatocyte maturation medium may thus also be HCM medium comprising or supplemented with the above-mentioned components. The hepatocyte maturation medium may thus also be HBM medium comprising or supplemented with the above-mentioned components. The hepatocyte maturation medium may thus also be Williams E based medium comprising or supplemented with the above-mentioned components.

In some embodiments, the hepatocyte maturation step preferably omits co-culture of the hepatic progenitor cells with any other cell type. In a specific aspect, the hepatocyte maturation step omits co-culture human umbilical vein endothelial cells (HUVEC) and/or mesenchymal stem cells (MSC) to produce a population of hepatocyte-like cells.

For differentiation into hepatocyte-like cells, hepatic progenitor cells are normally cultured for up to 14 days (e.g., up to 12 days) in the hepatocyte maturation medium as described above. The hepatic progenitor cells may, for example, be cultured in differentiation medium for about 12 to about 16 days (e.g., for about 12-14 days). In some embodiments, the hepatic progenitor cells are maintained in the cell culture vessel comprising an upper and lower chamber separated by a permeable membrane, optionally coated with at least one extracellular matrix protein, during maturation to hepatocyte-like cells, wherein the hepatic progenitor cells are in contact with the permeable membrane.

### II. In Vitro Cell Culturing Systems and Uses Thereof

Any of the hepatocyte-like cells produced by the methods of various aspects described herein (e.g., the methods of Section I) can be used in different applications where hepatocytes are required. Such hepatocyte-like cells are also within the scope of the present disclosure. For example, in some embodiments, the hepatocyte-like cells for use in the in vitro system described herein may have a normal BSEP gene. In some embodiments, the hepatocyte-like cells are unmodified hepatocyte-like cells (e.g., hepatocyte like-cells produced from wild-type PS cells) and may show a functional apico-basolateral polarity, transport of bile acids and/or de novo synthesis of bile acids.

In some aspect, provided herein is an in vitro cell culture system, which comprises a two-chamber cell culture vessel. In some embodiments, the cell culture vessel comprises:
(i) a cell culture vessel comprising an upper chamber and a lower chamber; wherein both the upper chamber and the lower chamber comprise a medium for culturing hepatocytes;
(ii) a permeable membrane separating the upper chamber and the lower chamber; and
(iii) a layer of hepatocyte-like cells grown on the permeable membrane.

In one aspect, the in vitro cell culture system comprises hepatocyte-like cells differentiated from a population of pluripotent stem cells having a modified ABCB11 gene. In some embodiments, the permeable membrane is optionally coated with at least one extracellular matrix protein, in a cell culture vessel with an upper and lower chamber separated by the permeable membrane. As noted above, suitable cell culture vessels are not particularly limited and can include any multi-well vessel comprising a permeable membrane as a barrier between wells or an insert may be added to a single well vessel thereby producing an upper and lower chamber separated by the permeable membrane. Suitable examples of permeable membranes include but are not limited to polycarbonate, polyester (PET), and collagen-coatedpolytetrafluoroethylene (PTFE).

Any of the in vitro cell culture system disclosed herein can be used, for example, to advance therapeutic discovery. Accordingly, provided herein include a method of screening for an agent for treating a cholestatic liver disease or determining the effect of a candidate agent on bile acid metabolism or transport are also provided herein.

The method comprises (i) providing an in vitro cell culture system as disclosed herein (ii) adding a bile acid (e.g., taurocholic acid (TCA) to the lower chamber, (iii) culturing the hepatocyte-like cells in the presence of a candidate agent; (iv) measuring the concentration of the bile acid in the upper chamber and/or in the lower chamber. In some embodiments, the candidate agent is identified the candidate agent as an agent for treating a cholestatic liver disease if the candidate agent changes the bile acid concentration determined in step (iv) as compared with the in vitro cell culture system in the absence of the candidate agent.

The candidate agents can be selected from the group consisting of proteins, peptides, nucleic acids (e.g., but not limited to, siRNA, anti-miRs, antisense oligonucleotides, and ribozymes), small molecules, nutrients (lipid precursors), and a combination of two or more thereof.

In some embodiments, effects of the candidate agents on the hepatocyte-like cells of the disclosure can be determined by measuring response of the cells and comparing the measured response with hepatocyte-like cells that are not contacted with the candidate agents. Various methods to measure cell response are known in the art, including, but not limited to, cell labeling, immunostaining, optical or microscopic imaging {e.g., immunofluorescence microscopy and/or scanning electron microscopy), spectroscopy, gene expression analysis, cytokine/chemokine secretion analysis, metabolite analysis, polymerase chain reaction (PCR), immunoassays, ELISA, gene arrays, spectroscopy, immunostaining, electrochemical detection, polynucleotide detection, fluorescence anisotropy, fluorescence resonance energy transfer, electron transfer, enzyme assay, magnetism, electrical conductivity (e.g., trans-epithelial electrical resistance (TEER)), isoelectric focusing, chromatography, immunoprecipitation, immunoseparation, aptamer binding, filtration, electrophoresis, use of a CCD camera, mass spectroscopy, or any combination thereof. Detection, such as cell detection, can be carried out using light microscopy with phase contrast imaging and/or fluorescence microscopy based on the characteristic size, shape and refractile characteristics of specific cell types.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1: Adaptive Transport of Bile Acids Induced by Loss of Bile Salt Export Pump Regulates Bile Acid Synthesis in Induced Hepatocytes

The goal of this study was to gain a greater understanding of the pathogenic mechanisms of genetic cholestatic liver diseases. Prominent among the subset of genetic diseases are defects in Bile Salt Export Pump (BSEP). Deficiency of this transporter is known to present in several clinical phenotypes, including Progressive Familial Intrahepatic Cholestasis type 2 (PFIC2), Benign Recurrent Intrahepatic Cholestasis type 2 (BRIC2), and Intrahepatic Cholestasis of Pregnancy (ICP). Strautnieks et al., Gastroenterology 134:1203-1214 (2008); and Strautnieks et al., Nature Genetics 20:233-238 (1998). PFIC2, the most severe form, has a wide spectrum of clinical manifestations—most commonly newborn cholestasis with varying rates of progression of the liver dysfunction. Nicolaou et al., Journal of Pathology 226:300-315 (2012). Patients with PFIC2 are also known to develop malignant transformation of hepatocytes during the first decade of life. Knisely et al., Hepatology 44:478-486 (2006). There are no therapeutic agents that have been found to be significantly effective for treatment of patients with severe PFIC2 because the specific alterations in the bile acid transport remain unclear.

To delineate the pathologic and compensatory alterations in BSEP deficient hepatocytes, several attempts have been made to generate rodent models that can recapitulate the phenotypes observed in patients with PFIC2. In the liver of the BSEP knock-out mouse, expression of ABCB1/MDR1, a transporter of bile acids at the bile canaliculus, is significantly increased, suggesting one compensatory mechanism to reduce the intracellular bile acid concentration via canalicular excretion. Wang et al., Hepatology 38:1489-1499 (2003). However, in analysis of gene expression of the human liver of patients with PFIC2, this MDR1 compensatory response was not evident. Keitel et al. Hepatology 41:1160-1172 (2005). Furthermore, since the bile of patients with PFIC2 contains a minimal amount of conjugated bile acids, BSEP deficient human hepatocytes seemingly lack the compensatory bile acid transporter on the canalicular membrane. Jansen et al., Gastroenterology 117:1370-1379 (1999).

Simple cultures of human hepatocytes fail to form functional apico-basolateral polarity, thus it has been difficult to investigate bile acid transport in human hepatocytes due to the lack of a suitable experimental system for dynamic tracing of transcellular transport of bile acids. Study of de novo bile acid synthesis by cultured hepatocytes has only been possible with a primary cell culture of explanted liver. Because an explanted liver from patients with PFIC2 is rarely available, an experimental investigation into the regulatory mechanism of bile acid synthesis and transport in human BSEP deficient hepatocytes has not been possible.

To overcome this difficulty, the present study used human induced pluripotent stem cells (iPSCs) and developed an in vitro culture system where iPSCs were differentiated into hepatocyte-like cells on a permeable membrane of a two-chamber (Transwell) system. The in vitro culture system disclosed in the Example here is an improvement of the in vitro system disclosed in Asai et al., Development 144:1056-1064 (2017), wherein inter alia, the instant in vitro culture system provides a disease model produced with a single population of cell, i.e., does not require co-culture with other cell types. Using this system, the present study investigates the fate of intracellular bile acids and their role as a mediator between de novo bile acid synthesis and transcellular transport.

Taken together, the instant study has provided an in vitro disease model for BSEP deficiency. The results reported herein provide new insights into molecular mechanisms that underlie the pathophysiology of BSEP deficiency and provide targets for therapeutic intervention in patients with PFIC2.

### Methods

### Genotype Selection and Description of the Index Case

Deleterious mutations of BSEP/ABCB11 were searched in a cohort of patients with progressive familial intrahepatic cholestasis type 2 (PFIC2). The patients in the cohort of this study had compound heterozygous mutation in BSEP, including R1090X and R928X; both are nonsense truncating mutations. One set of siblings who had an identical genotype of ABCB11; c.2782 C>T (R928X) and c.3268 C>T (R1090X) were identified. Because their parents were heterozygous for each truncating mutation, the genetic test indicates compound heterozygous mutations. Both siblings presented with severe cholestasis and required liver transplant before age of 1 year. To investigate the biological impact of a severe mutation in bile acid efflux, the R1090X truncating nonsense mutation was selected, which was reported in previous cases as a homozygous genotype. Strautnieks et al., Gastroenterology 134:1203-1214 (2008); Strautnieks et al., Nature Genetics 20:233-238 (1998); and Zhou et al., Journal of Proteome Research 14:4844-4850 (2015). Liver tissues from the subject were obtained from the explanted liver.

Cell Culture and Differentiation of iPSCs to Hepatocyte-Like Cells

All chemical materials were purchased from Sigma-Aldrich (St. Louis, Mo.) unless otherwise indicated. All cells were incubated at 37° C. in a humidified 5% CO₂. The iPSCs (clone code: 1383D6) were derived from a healthy donor with thorough characterization of pluripotency and karyotype. Takayama et al., Hepatology Commun 1:1058-1069 (2017). Protocols for endoderm differentiation, hepatic specification, and hepatocyte maturation are modified from previously described protocols. Asai et al., Development 144:1056-1064 (2017). Briefly, for definitive endoderm differentiation, iPSCs were dissociated with Accutase and plated onto a Laminin 511 (Matrixsome, Osaka, Japan) coated cell culture dish. The medium was replaced with RPMI1640 (ThermoFisher, Waltham, Mass.) containing 2% B27 (ThermoFisher), 1 mM sodium butyrate (for the first 3 days), Wnt3a 50 ng/mL (R&D systems, Minneapolis, Minn.) and Activin 100 ng/mL (R&D) for 6 days. For hepatic specification, cells were further treated with FGF2 10 ng/mL (R&D) and BMP4 20 ng/mL (R&D) for 3 days. Cells were dissociated with TrypLE (ThermoFisher) and were plated on the membrane of Transwell insert (Corning, Corning, N.Y.). Then, cells were cultured in Hepatocyte Culture Medium (HCM) (Lonza, Allendale, N.J.) for 12 days. HCM is supplemented with HCM BulletKit (Lonza): transferrin, hydrocortisone, BSA-fatty acid free (BSA-FAF), ascorbic acid, insulin, GA-1000, and omitting human epidermal growth factor. 10 ng/mL recombinant hepatocyte growth factor (HGF), 100 nM dexamethasone, and 5% of fetal bovine serum (ThermoFisher) were added to supplement HCM.

In order to monitor the efficiency of the hepatic differentiation, the albumin production measured by ELISA assays of the culture supernatant of the hepatocyte-like cells were quantified two days prior to the experiments. HGF was removed from the medium 3 days prior to the experiments when indicated. The general scheme for producing hepatocyte-like cells from iPS cells is shown in Table 1 below.

TABLE 1
Differentiation Scheme

| | | | | |
|---|---|---|---|---|
| Reagent | Storage | Stock Conc. | Final Conc. | Volume (per 1 ml) |

Endoderm differentiation medium 1 - Day 1 (On day 1, iPSCs were dissociated with
Accutase (modified trypsin) and re-plated on a regular plastic culture dish coated with
Laminin 511 or Matrigel).

| RPMI + HEPES | 4 C. | N/A | | N/A | | 1 | ml |
|---|---|---|---|---|---|---|---|
| B27 (+insulin) | −20 C. | X50 | | | | 20 | ul |
| Activin A | −80 C. | 100 | ng/ul | 100 | ng/ml | 1 | ul |
| Wnt3a | −80 C. | 50 | ng/ul | 50 | ng/ml | 1 | ul |
| Y 27632 | −80 C. | 10 | mM | 10 | uM | 1 | ul |

Endoderm differentiation medium 2 - Day 2

| RPMI + HEPES | 4 C. | N/A | | N/A | | 1 | ml |
|---|---|---|---|---|---|---|---|
| B27 (+insulin) | −20 C. | X50 | | | | 20 | ul |
| Activin A | −80 C. | 100 | ng/ul | 100 | ng/ml | 1 | ul |
| Wnt3a | −80 C. | 50 | ng/ul | 50 | ng/ml | 1 | ul |
| Sodium Butyrate | −80 C. | 500 | mM | 500 | uM | 1 | ul |

Endoderm differentiation medium 3 - Day 3 and Day 4

| RPMI + HEPES | 4 C. | N/A | | N/A | | 1 | ml |
|---|---|---|---|---|---|---|---|
| B27 (+insulin) | −20 C. | X50 | | | | 20 | ul |
| Activin A | −80 C. | 100 | ng/ul | 100 | ng/ml | 1 | ul |
| CHIR99021 | −80 C. | 20 | mM | 3 | uM | 0.15 | ul |
| Sodium Butyrate | −80 C. | 500 | mM | 500 | uM | 1 | ul |

Endoderm differentiation medium 4 - Day 5 (on this day, cells are dissociated with trypsin
and re-plated on a permeable membrane of a Transwell)

| | | | | | |
|---|---|---|---|---|---|
| RPMI + HEPES | 4 C. | N/A | N/A | 1 | ml |

Endoderm differentiation medium 1 - Day 1 (On day 1, iPSCs were dissociated with
Accutase (modified trypsin) and re-plated on a regular plastic culture dish coated with
Laminin 511 or Matrigel).

| B27 (+insulin) | −20 C. | X50 | | | | 20 | ul |
|---|---|---|---|---|---|---|---|
| Activin A | −80 C. | 100 | ng/ul | 100 | ng/ml | 1 | ul |
| CHIR99021 | −80 C. | 20 | mM | 3 | uM | 0.15 | ul |
| Y 27632 | −80 C. | 10 | mM | 10 | uM | 1 | ul |

Endoderm differentiation medium 5 - Day 5

| RPMI + HEPES | 4 C. | N/A | | N/A | | 1 | ml |
|---|---|---|---|---|---|---|---|
| B27 (+insulin) | −20 C. | X50 | | | | 20 | ul |
| Activin A | −80 C. | 100 | ng/ul | 100 | ng/ml | 1 | ul |
| CHIR99021 | −80 C. | 20 | mM | 3 | uM | 0.15 | ul |

Hepatic specification medium - Day 7, 8, and 9

| RPMI + HEPES | 4 C. | N/A | | N/A | | 1 | ml |
|---|---|---|---|---|---|---|---|
| B27 (+insulin) | −20 C. | X50 | | | | 20 | ul |
| bFGF | −80 C. | 100 | ug/ml | 50 | ng/ml | 0.5 | ul |
| BMP4 | −80 C. | 50 | ug/ml | 20 | ng/ml | 0.4 | ul |

Hepatocyte maturation medium - Day 10-22

| HBM Basal Media + | | | | | | 1 | ml |
|---|---|---|---|---|---|---|---|
| HCM* | | | | | | | |
| FBS | −20 C. | | | 5% | | 50 | ul |
| Dexamethasone | −80 C. | 2.5 | mM | 0.1 | uM | 0.04 | ul |
| HGF | −80 C. | 50 | ug/ml | 20 | ng/ml | 0.4 | ul |

*HCM Hepatocyte Culture Media (Lonza CC-3198) and uses all components according to the bullet kit reipe but omits the EGF. FBS (Fetal bovine serum): complement heat inactivated.

CRISPR/Cas9 Genome Editing of Human iPSCs

CRISPR/Cas9 was used to introduce the truncating mutation of BSEP/ABCB11 in 1383D6 iPSCs. Candidate sgRNA target sites were selected according to the on- and off-target prediction scores from the web-based tool, CRISPOR (http://crispor.org/). The selected sgRNAs were cloned into the pX458M-HF vector that was modified from the pX458 vector (addgene #48138) and carried an optimized sgRNA scaffold and a high-fidelity Cas9 (eSpCas9 1.1)-2A-GFP expression cassette. The editing activity of the plasmid was validated in 293T cells by T7E1 assay. Kumar et al., Plos One 5 (2010); Chen et al., Cell 155:1479-1491 (2013); and Aymaker et al., Science 351:84-88 (2016). A phosphorothioated single stranded oligonucleotide-DNA (ssODN) was designed to include the intended mutations, silent mutations (to block sgRNA retargeting and to create a new restriction enzyme site for genotyping), and homologous sequence. A single cell suspension of iPSCs was prepared using Accutase and 1×10^{e6} cells were nucleofected with 2.5 μg of the plasmid and 2.5 μg of ssODN using program CA137 (Lonza). Forty-eight hours later, transfected cells were sorted one cell per well into 96 well plates based on the GFP expression. The cell clones were expanded and selected by a screening of restriction enzyme digestion. The correctly edited clones were selected based on the gain of the restriction enzyme sites on both alleles and further confirmed by Sanger sequencing for identification of bi-allelic single nucleotide mutations. Cell clones that went through the same targeting process but remained unedited were expanded and used as isogenic parental controls.

### Measurement of Bile Acid Concentration in Culture Medium

The concentration of total bile acid in culture supernatant was determined by Diazyme TBA assay (Diazyme Laboratories, Poway, Calif.) following the manufacturer's instructions. For tracer experiments, stable isotope labelled taurocholic acid (sodium taurocholic acid, [2, 2, 4, 4-²H₄]TCA, here referred to as D4-TCA)) was purchased from Cambridge isotope laboratories (Tewksbury, Mass.). For long term transport assay, D4-TCA was added into the culture medium in the lower chamber at 1 μM and 10 μM. After incubation, the supernatant of upper and lower chambers was collected. For uptake and washout experiments, cells were incubated with buffer containing 118 mM NaCl, 23.8 mM NaHCO₃, 4.83 mM KCl, 0.96 mM KH₂PO₄, 1.20 mM MgSO₄, 12.5 mM HEPES, 5 mM glucose and 1.53 mM CaCl₂. After 15 minutes of pre-incubation, D4-TCA (10 μM) containing buffer was added to the lower chambers. For uptake experiments, at 5 min and 15 min, cells were collected and frozen. For sodium-free buffer, sodium was replaced by choline (choline chloride or choline bicarbonate). For washout experiments, after 1 h of incubation with D4-TCA containing buffer, cells were washed with buffer and placed in a fresh buffer. The supernatant was then collected at 5 min, 15 min, 30 min, and 60 min from the upper and lower chamber separately. The MRP4 inhibitor, Ceefourin1, was purchased from Abcam (Cambridge, Mass.). All the samples received a fixed amount of D4-TCDCA as an internal standard and purified by protein precipitation with Acetonitrile. A calibration curve of D4-TCA was constructed using D4-TCDCA as internal standard for quantification of D4-TCA in samples. In some experiments, the endogenous bile acids and D4-TCA concentrations were measured at the University of Tokyo after confirming the compatibility of both methods.

### Measurement of D4-TCA and Endogenous Bile Acids Concentrations by Liquid Chromatography-Mass Spectrometry (LC-MS)

Cells on membrane lysed with 500 μL methanol and buffer from upper and lower chamber were subjected to LC-MS/MS analysis to quantify the concentration of D4-TCA and endogenous bile acids. 30 μL of the prepared samples were transferred to a 1 mL 96-well plate and then mixed with 120 μL of internal standard solution (100 nM D8-TCA, Santa Cruz Biotechnology, Santa Cruz, Calif.) in methanol or D5-TCA (Toronto Research Chemicals, North York, Canada) in acetonitrile. After vortex mixing, the mixtures were filtered using FastRemover for Protein (GL Sciences, Tokyo, Japan) and transferred to 96-well plate for LC-MS/MS analysis. The sample analysis was conducted on a SCIEX 5500 tandem mass spectrometer (Applied Biosystems/MDS SCIEX, Toronto, Canada) equipped with a Prominence LC system (Shimadzu, Kyoto, Japan), and operated in electrospray ionization mode. For measurement of D4-TCA concentration, samples were injected onto a CAPCELL PAK C18 MGM column (2 mm i.d.×50 mm, Shiseido, Tokyo, Japan) and separated with the following gradient program: 10% B for 0.3 min, 10-90% B for 1.7 min, 90% B for 1.3 min, 90-10% B for 0.1 min, and 10% B for 1.9 min. The total flow rate was 0.4 mL/min, the mobile phase was 5 mM ammonium acetate in water (A) and methanol (B), and the column temperature was maintained at 40° C. For measurement of endogenous TCA concentrations, samples were injected onto a ACQUITY UPLC BEH C18 column (2.1 mm i.d.×150 mm, Waters, Milford, Mass.) and separated with the following gradient program: 20% B for 0.5 min, 20-70% B for 10 min, 70-98% B for 0.1 min, 98% B for 0.4 min, 98-20% B for 0.1 min and 20% B for 0.9 min. The total flow rate was 0.5 mL/min, the mobile phase was 0.1% formic acid in water (A) and 0.1% formic acid in acetonitrile (B), and the column temperature was maintained at 60° C. The mass spectrometer was operated in negative multiple reaction monitoring (MRM) mode. All peak integration and data processing were performed using SCIEX Analyst (Applied Biosystems/MDS SCIEX).

### Analysis of Endogenous TCA Concentrations by Liquid Chromatography-Mass Spectrometry (LC-MS)

Quantitative analysis of endogenous taurocholic acid (TCA) in the culture medium was carried out by stable-isotope dilution LC-MS with electrospray ionization in single ion recording (SIR-MS) negative ion mode using a Waters TQ-XS triple quadruple mass spectrometer interfaced with Aquity UPLC system (Milford, Mass.). Quantification of TCA was achieved by interpolation of the area ratio of each bile acid to its corresponding stable-labeled analog against a calibration curve of known concentrations of bile acids. After exchanging culture medium, cells were incubated for 48 h. The supernatants from the upper and lower chambers were collected separately. The culture supernatants and cell lysates were extracted with reverse phase solid-phase cartridge and bile acids (synthesized TCA and exogenous D4-TCA) were quantified using each standard.

### Transmission Electron Microscopy

The monolayer cells on the Transwell membrane were fixed with 2% paraformaldehyde, 2.5% glutaraldehyde in 0.1 mol/L cacodylate, pH 7.2 for 1 hour at 4° C. Specimens were then post-fixed with 1% OsO₄ for 1 hour, dehydrated in an ethanol series (25, 50, 75, 95, and 100%), and infiltrated with dilutions of ETOH/LX-112 and then embedded in LX-112 (Ladd Research Industries, Williston, Vt.) while still on the culture membrane surface. Blocks were polymerized for 3 days at 60° C. The monolayer was ultra-thin sectioned on Reichert EM UC7 ultra-microtome (Depew, N.Y.), perpendicular to the plane of the Transwell membrane and mounted on grids, which were post-stained with uranyl acetate and lead citrate. The sections were viewed using a Hitachi H7650 electron microscope (Tarrytown, N.Y.).

### Microscopic Imaging and 3D Image Reconstruction

Immunofluorescence and light microscopy imaging were performed using an Olympus microscope and DP71 camera (Olympus, Center Valley, Pa.) and Zeiss LSM710 confocal microscope (San Diego, Calif.). 3D image reconstruction of z-stack confocal images was generated using Imaris Version 7.7 software (Bitplane, Concord, Mass.).

### Protein Quantification

Unless specified, supernatant of upper and lower chamber is collected separately at 24 hours after last medium exchange. Human albumin in the collected culture supernatant was quantified with ELISA kit (Bethyl Laboratories, Montgomery, Tex.) following the manufacture instruction. For western blotting, the cells were lysed with lysis buffer (Cell Signaling Technology, Cambridge, Mass.) with proteinase and phosphatase inhibitor cocktail. Protein extracts were resolved by 4-12% SDS-PAGE and transferred to PVDF membranes. Membranes were blocked in diluted skim milk and incubated with primary antibodies at 4° C. overnight. Membranes were then washed and incubated with the secondary antibodies for 1 h at room temperature and washed again, followed by incubation of chemiluminescence reagents. Images were captured using Chemi-doc system (Bio-Rad).

### Quantitative PCR

Total RNA was extracted from cells by the RNeasy kit (Quiagen) following the manufacturer's instructions. After measuring total RNA concentration, 500 ng of RNA were subjected to reverse transcription reactions. The real-time PCR by TaqMan probe system (gene expression master mix) and the QuantStudio system (ThermoFisher) quantified mRNA of target genes, with specific primers and quantification protocol. After normalized with a housekeeping gene (18S rRNA), each gene expression level was described relative to normal i-Hep or baseline controls.

### Statistics

All in vitro experiments were performed at least in triplicate. Experimental values are expressed as mean±SEM, and statistical significance was determined by 2-tailed Student's t test or by 2-way ANOVA for comparison between 3 or more groups, followed by Bonferroni's multiple comparison post-hoc test with a significance set at p<0.05. Statistical analysis and graphic description were performed by GraphPad Prism (GraphPad Software).

### Results

(i) Generation of BSEP/ABCB11^{R1090X} Mutant Human iPSCs.

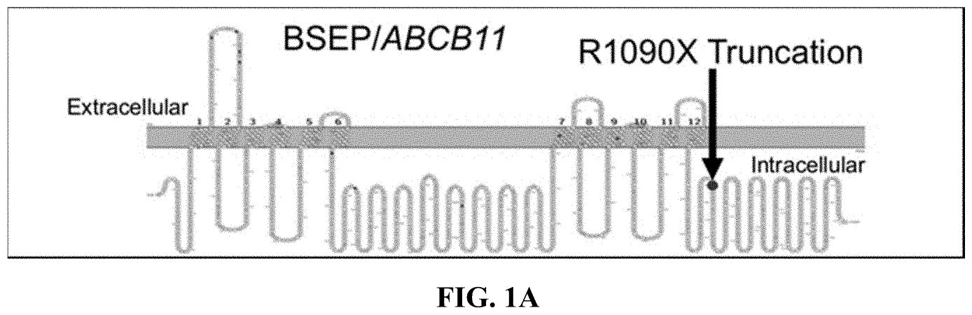

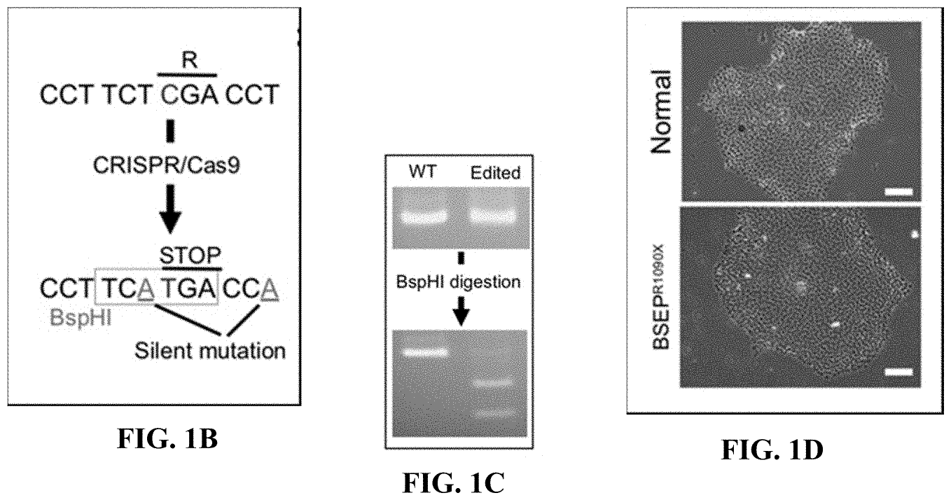

To elucidate the specific effects of R1090X truncating mutation on the BSEP function in hepatocytes, CRISPR-Cas9 genome editing was used to target the R1090 codon in the BSEP/ABCB11 gene in iPSCs obtained from a healthy donor (FIG. 1A). A single stranded oligonucleotide-DNA (ssODN) was designed to replace the codon of CGA (arginine) at position 1090 with TGA (stop codon) as well as two silent mutations to create de novo BspH1 restriction sites to facilitate colony screening (FIG. 1B). After transfection of the CRISPR-Cas9-2A-GFP plasmid and ssODN, GFP⁺ cells were FACS sorted and clones were established. Correctly targeted clones were identified by BspH1 digestion and the introduction of homozygous R1090X mutation was confirmed by Sanger sequencing (FIG. 1C). To evaluate whether the CRISPR manipulation affected their pluripotency of iPSCs, size and shape of their colonies were monitored. Parental and R1090X edited iPSCs showed comparable colony morphology. The expression of OCT4, a marker of pluripotent cells, remained comparable in parental and R1090X edited iPSCs.

(ii) BSEP^{R1090X} iPSCs Differentiate into Hepatocyte-Like Cells and Express BSEP Protein in an Altered Pattern.

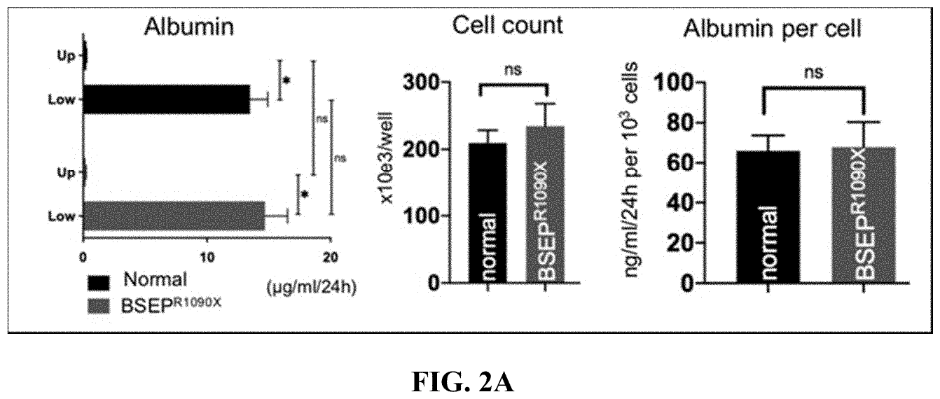

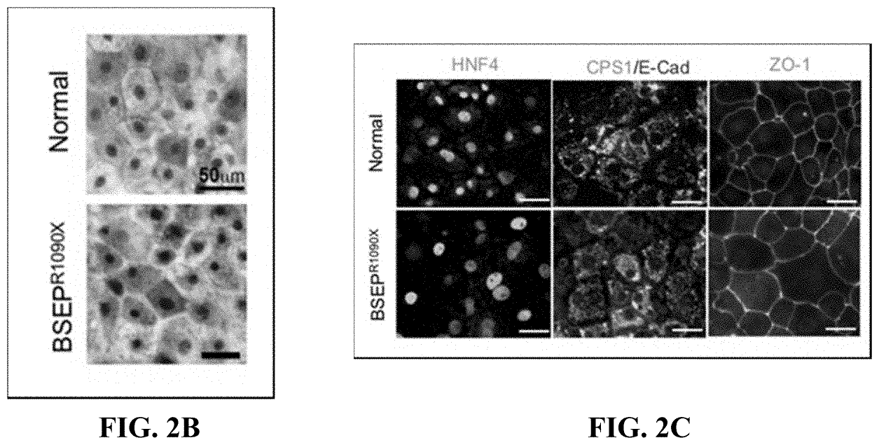

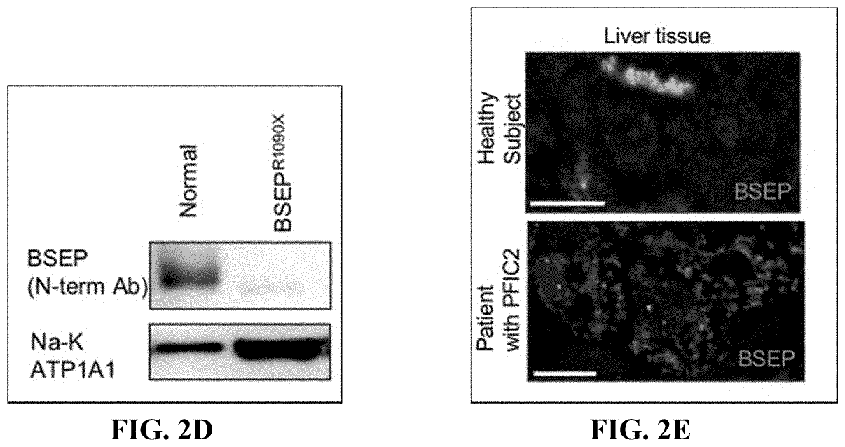

To determine whether the edited iPSCs-BSEP^{R1090X} are able to differentiate into hepatocytes, hepatic differentiation was first induced with the same method as the parental iPSCs with normal BSEP (iPSCs-BSEPnormal or normal iPSCs). To quantify the efficiency of the hepatic differentiation, the albumin secretion of induced hepatocytes was measured (i-Hep). The BSEP^{R1090X} hepatocytes (BSEP^{R1090X} i-Hep) exhibited comparable albumin secretion into the culture medium to the normal i-Hep (FIG. 2A). Most of the albumin was secreted into the lower chamber (FIG. 2A, left panel). The number of cells in a well and albumin production per cell were comparable between normal and BSEP^{R1090X} i-Hep (FIG. 2A, center and right panels). Both i-Hep showed polygonal hepatocyte-like cells with occasional bi-nuclei formation (FIG. 2B). At the final stage of the differentiation protocol, BSEP^{R1090X} i-Hep expressed hepatic differentiation markers (HNF4a, CPS1) and tight junction protein (ZO1) in a pattern comparable to that of normal i-Hep (FIG. 2C). To determine the pattern of cellular polarity, a co-immunostaining of i-Hep with F-actin was performed (relatively concentrated on the canalicular membrane of hepatocytes in the human liver tissue), Na—K transporting ATPase al (ATP1A1: expressed on the basolateral membrane in hepatocytes), and ZO1 (expressed between the canalicular and basolateral membrane) and analyzed their z-stack confocal images. F-actin was detected mainly on the apical membrane in both normal and BSEP^{R1090X} i-Hep, with a lower degree of expression on the lateral membrane. ATP1A1 was detected on the lateral membrane, while the basal membrane was not depicted by our confocal microscope settings due to the optical interference of the Transwell membrane. ZO1 was detected at the corner of the cells where apical and lateral membranes meet. These results indicate intact cellular polarity in both normal and BSEP^{R1090X} i-Hep.

Next, to determine whether genomic editing of the ABCB11 gene alters the BSEP protein expression pattern, western blotting and immunofluorescent staining of BSEP was performed. An antibody targeting the N-terminus of the protein detected BSEP in both normal and BSEP^{R1090X} i-Hep, though expression was clearly lower in BSEP^{R1090X} (FIG. 2D). The molecular weight of the BSEP^{R1090X} was lower than the normal BSEP, indicating a truncation of the BSEP. Immunostaining with the same N-terminal antibody revealed that the BSEP^{R1090X} i-Hep expressed BSEP protein in an aberrant pattern. While normal i-Hep expressed BSEP mainly at the apical membrane of monolayer cells, BSEP was localized in the cytosol in a dot-like pattern in BSEP^{R1090X} i-Hep. To determine whether the pattern of BSEP expression reflects the cellular localization in liver tissue of patients with PFIC2, immunofluorescent staining of liver biopsy specimens using the same N-terminal antibody was performed (FIG. 2F). Compared to hepatocytes obtained from the liver of a healthy subject, where BSEP is localized at a canalicular membrane structure, BSEP in hepatocytes of the patients with PFIC2 was localized in the cytosol in a clustering pattern.

These results indicate that genomic editing of the ABCB11 gene in iPSCs results in the aberrant localization of BSEP in i-Hep, comparable to the pattern of BSEP localization seen in the hepatocytes in the liver of patients with PFIC2.

(iii) Cellular Ultrastructure in BSEP^{R1090X} i-Hep Recapitulates Hepatocyte Abnormalities in the Liver Tissue of the Patient with PFIC2.

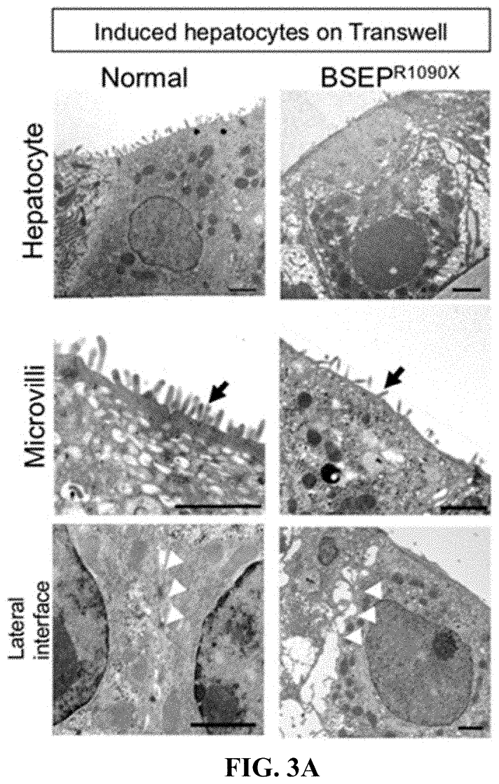

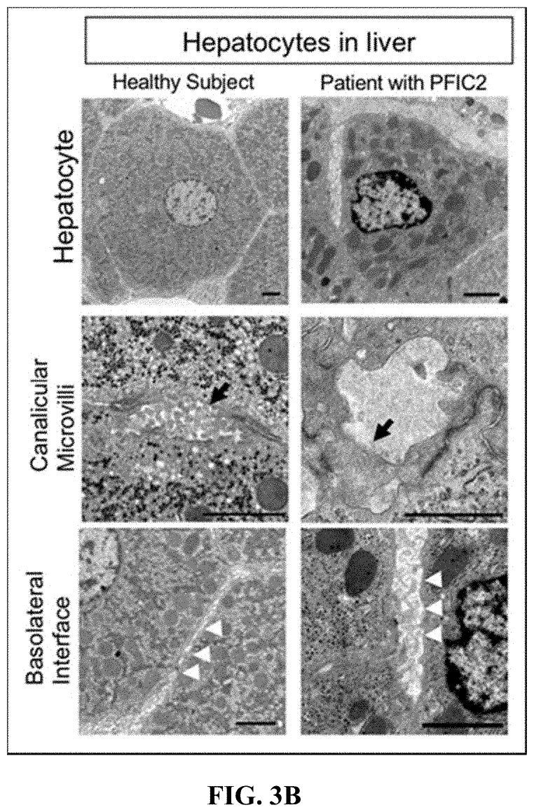

It has been reported that a development of microvilli on the bile canaliculus depends on export of bile acid across the canalicular membrane of hepatocytes. Bove et al., Pediatr Devel Pathol 7:315-334 (2004); Wolf-Pesters et al., Tissue Cell 4:379-388 (1972); and Gallin et al., Microsc Res Techniq 39:406-412 (1997). To investigate the effect of the altered BSEP expression pattern, morphological analysis of i-Hep derived from normal and BSEP^{R1090X} iPSCs was performed. To assess ultrastructural changes, i-Hep at the last stage of differentiation were evaluated by electron microscopy (FIG. 3A). Normal i-Hep showed a monolayer structure with dense microvilli on the apical membrane. These findings indicate that i-Hep developed epithelial polarization as a monolayer on the Transwell membrane, directing the apical membrane toward the upper chamber and basal interface toward the lower chamber via the permeable membrane of the Transwell. BSEP^{R1090X} showed fewer microvilli on their apical surface, indicating reduced bile acid transport across the apical membrane. Irregularity of the basolateral membrane in BSEP^{R1090X} with wider interstitial space between hepatocytes were also found. To determine whether these ultrastructural features are relevant to the patient with PFIC2 (R1090X mutation), the liver explant obtained at the time of liver transplant was investigated via electron microscopy (FIG. 3B). Compared to hepatocytes from a normal liver, hepatocytes from the patients with PFIC2 exhibited a decreased number of microvilli in the bile canaliculus and wider interstitial space between basolateral membranes of adjacent cells.

These corresponding morphological features between i-Hep and liver suggest that the pathological process of the patient with the truncating mutation manifests in BSEP^{R1090X} i-Hep.

(iv) BSEP^{R1090X} is Deficient in Exogenous Bile Acid Transport Via the Basolateral-to-Apical Phase.

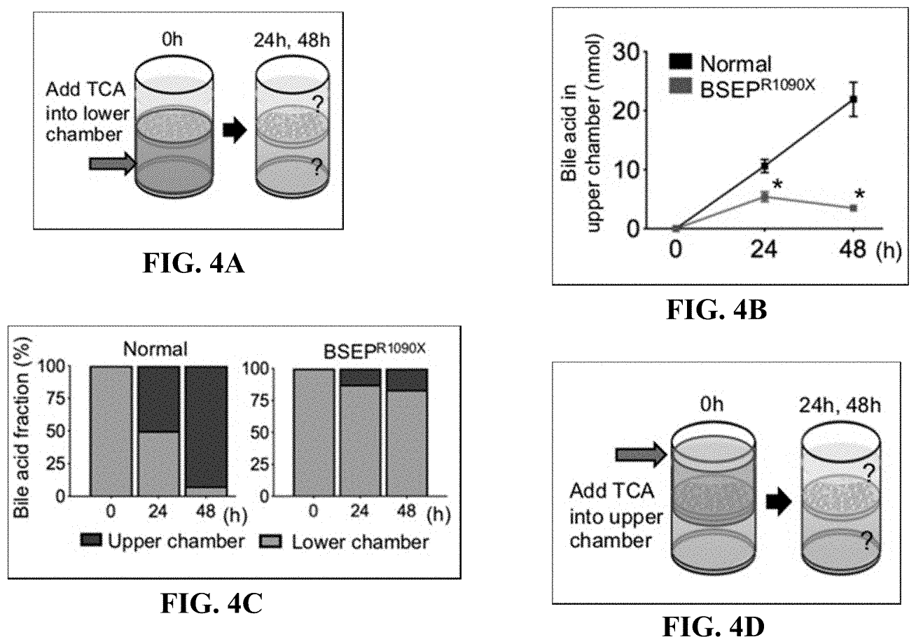

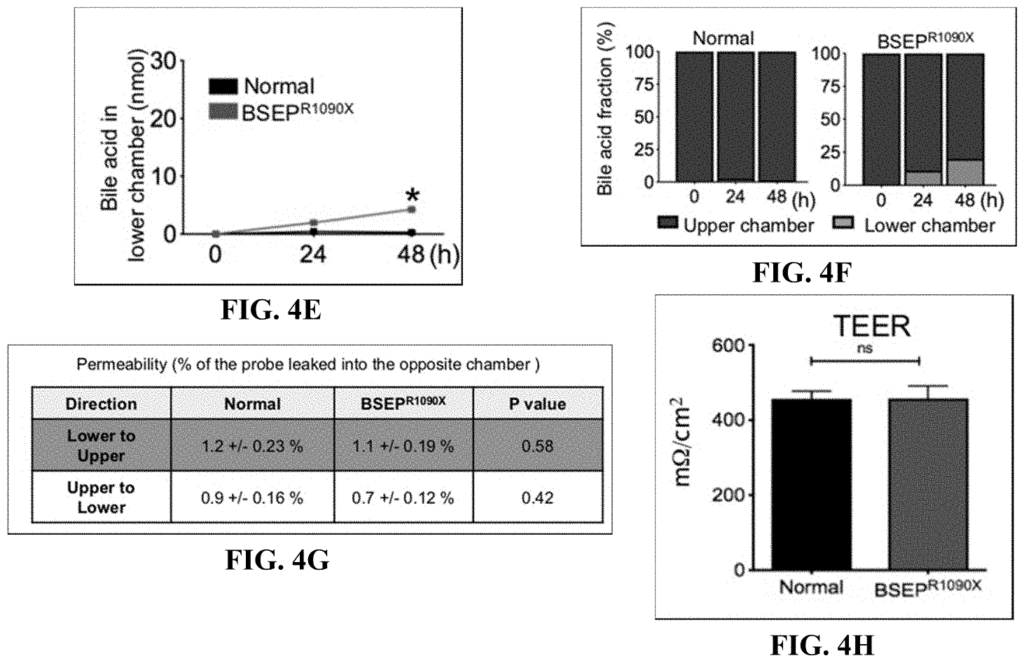

The structural defect of microvilli on the apical surface on BSEP^{R1090X} i-Hep suggested compromised canalicular function, specifically in bile acid export. To examine how the BSEP truncation impacts the exporting function of BSEP in response to exogenous bile acids, the capability of bile acid transport of i-Hep was evaluated by adding TCA to the lower chamber. In a previous study, normal i-Hep transport bile acids from the basolateral phase to apical phase was demonstrated (transcellular transport from sinusoid to bile canaliculus). To assess whether BSEP^{R1090X} i-Hep manifest altered transcellular transport of conjugated bile acids, the amount of TCA in culture medium from the upper chamber 24 hours and 48 hours after loading TCA in the lower chamber was measured. In normal i-Hep, the amount of transported TCA in the upper chamber increased at 24 h with the majority of the loaded TCA traversing from the lower to the upper chamber by 48 h (FIGS. 4A-4C). In contrast, in BSEP^{R1090X}, most of the loaded TCA remained in the lower chamber. This data formed the first indication that the transporting direction of conjugated bile acids in BSEP^{R1090X} i-Hep differs from the direction seen in normal i-Hep.

To determine whether this direction of exogenous TCA transport is specific to a basolateral-to-apical direction, the amount of TCA in the lower chamber after loading it into the upper chamber was measured (FIGS. 4D-4F). In both normal and BSEP^{R1090X}, most of the loaded TCA remained in the upper chamber. To measure the degree of paracellular “leak” of TCA, the permeability of the monolayer in normal and BSEP^{R1090X} i-Hep was compared (FIG. 4G). After 48 hours, a minimal, comparable amount of the fluorescent probe (10,000 MW dextrose conjugated Alexa-fluoro) was transported from the lower to upper chamber in both normal and BSEP^{R1090X}. Furthermore, to compare their barrier function as a monolayer, trans-epithelial electrical resistance (TEER) between the upper and lower chamber was measured (FIG. 4H). The resistance of the BSEP^{R1090X} monolayer was comparable to the normal i-Hep monolayer.

Together, these results demonstrate that BSEP^{R1090X} i Hep exert a specific deficiency in basolateral-to-apical transcellular transport of conjugated bile acids.

(v) Intracellular TCA in BSEP^{R1090X} i-Hep Remains Comparable to Normal i-Hep During Transcellular Transport of TCA.

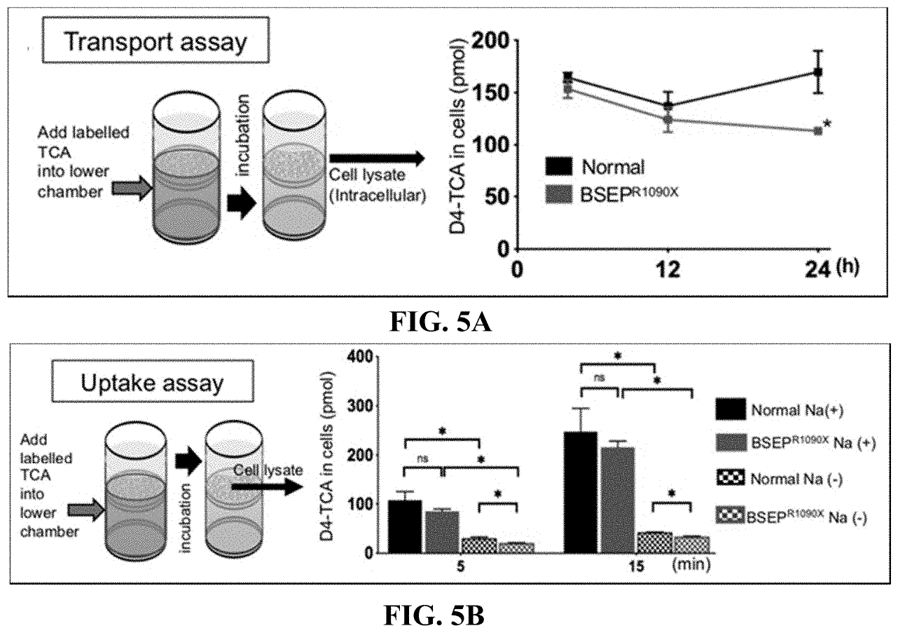

To test whether decreased bile acid export induces intracellular accumulation of TCA in BSEP^{R1090X}, molecular tracing experiments and quantified TCA concentration in cell lysates after loading TCA into the lower chamber was performed. By using isotope labelled TCA (D4-TCA), the export and uptake of TCA executed by i-Hep independent of endogenous TCA was measured. First, whether BSEPR1090X i-Hep accumulate more intracellular TCA than normal i-Hep when exposed to exogenous TCA from the lower chambers was determined (FIG. 5A). To quantify the long-term transport activity, a 24-hour tracing experiment was performed. After loading D4-TCA in the lower chambers (1 μM), the amount of D4-TCA in the cell lysates was quantified at 4, 12, and 24 hours. At each time point in BSEP^{R1090X} i Hep, the cell lysates contained comparable (4 h and 12 h) or smaller amount (24 h) of D4-TCA compared to the normal i-Hep. This result demonstrates that BSEP^{R1090X}i-Hep do not accumulate intracellular TCA to a greater degree than the normal i-Hep despite having decreased apical export of TCA.

The result prompted a test of whether BSEP^{R1090X}i-Hep have a comparable capability to uptake D4-TCA from the lower chamber by measuring intracellular D4-TCA after a short period of time before reaching their saturation. At a physiological dose of TCA (10 μM) in the culture medium in the lower chamber, normal and BSEP^{R1090X} i-Hep showed comparable amount of uptake at 5 min and 15 min of the incubation time (FIG. 5B). To test whether D4-TCA uptake was sodium dependent, intracellular D4-TCA after incubating with sodium depleted culture medium was measured. In both normal and BSEP^{R1090X} i-Hep, the intracellular D4-TCA was significantly lower compared to that in the condition of sodium containing regular culture medium. These results indicate that BSEP^{R1090X}i-Hep exhibit comparable capability of TCA uptake in a sodium dependent fashion.

Together, the accumulation of intracellular TCA in BSEP^{R1090X} i-Hep was comparable in normal i-Hep in the setting where they uptake comparable amount of the conjugated bile acid across the basolateral surface into the intracellular space, while having deficient export of TCA across the apical membrane.

(vi) BSEP^{R1090X} i-Hep Export Intracellular TCA Via the Basolateral Membrane Toward the Lower Chamber.

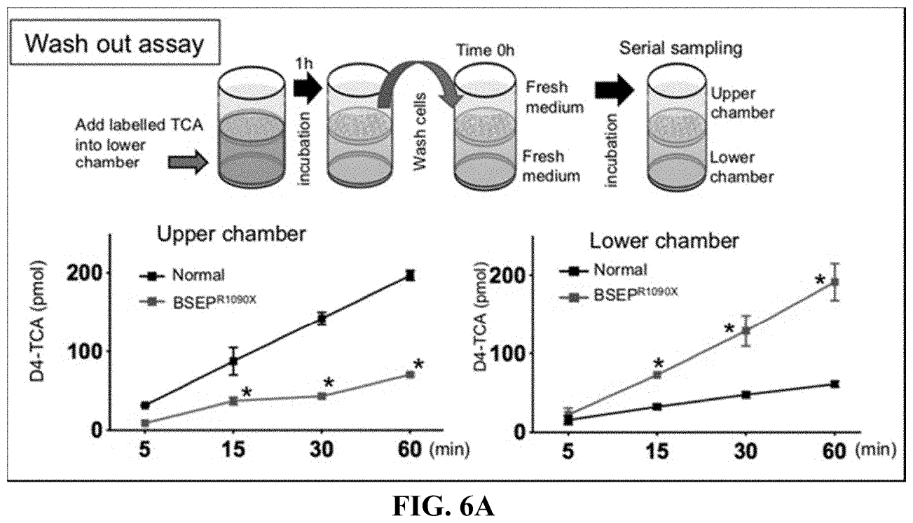

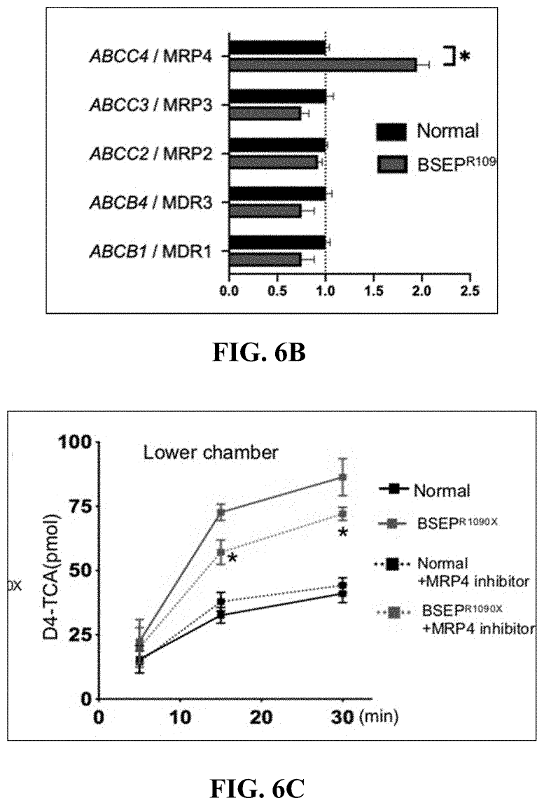

Because BSEP^{R1090X} i-Hep have a limited capacity for apical export of TCA while taking up comparable amounts of TCA, these results suggested that BSEP^{R1090X} compensates via other export channels, potentially basolateral export. To determine whether BSEP^{R1090X} i-Hep export intracellular TCA via the basolateral membrane after uptake of TCA, a “wash-out” tracing experiment with D4-TCA was performed. After one hour of incubation for uptake of D4-TCA from the lower chamber, i-Hep cells were washed gently with medium and incubated in fresh culture medium. At 5, 15, 30 and 60 minutes, D4-TCA was quantified in the upper and lower chamber to determine their export rates from the apical and basolateral membrane, respectively (FIG. 6A). The BSEP^{R1090X} i-Hep showed increased export into the lower chamber compared to normal i-Hep at each time point. In addition, BSEP^{R1090X} showed greater export toward the lower chamber than export toward the upper chamber, as seen at longer time points. The normal i-Hep showed the opposite export pattern when compared to BSEP^{R1090X} i-Hep. These results indicate that BSEP^{R1090X}i-Hep utilize basolateral export of intracellular TCA when their apical export is deficient.

To identify transporters on the basolateral membrane of BSEP^{R1090X} i-Hep, gene expressions were profiled of the transmembrane ATP Binding Cassette (ABC) transporters by quantitative RT-PCR. A gene up-regulation was found of ABCC4/MRP4—known to transport conjugated bile acids, including TCA (FIG. 6B). To further determine the functional role of MRP4 in the basolateral export of BSEP^{R1090X} i-Hep, washout tracing experiments with and without the MRP4 inhibitor (Ceefourin1) in the culture medium was performed. Cheung et al., Biochemical Pharmacology 91:97-108 (2014); and Jördens et at, Glia 63:2092-2105 (2015). Ceefourin1 decreased basolateral export of TCA in BSEP^{R1090X} i-Hep while it did not alter the basolateral export in the normal i-Hep (FIG. 6C). These results indicate that MRP4 plays a role in intracellular-to-basolateral export of TCA in BSEP deficiency. Together, when exposed to exogenous TCA, the instant study has demonstrated that BSEP^{R1090X} i-Hep maintain low intracellular TCA concentration by export via basolateral membrane transporter(s).

(vii) Maturing BSEP^{R1090X} i-Hep Adapt an Alternative Export of Newly Synthesized Bile Acids Via the Basolateral Membrane.

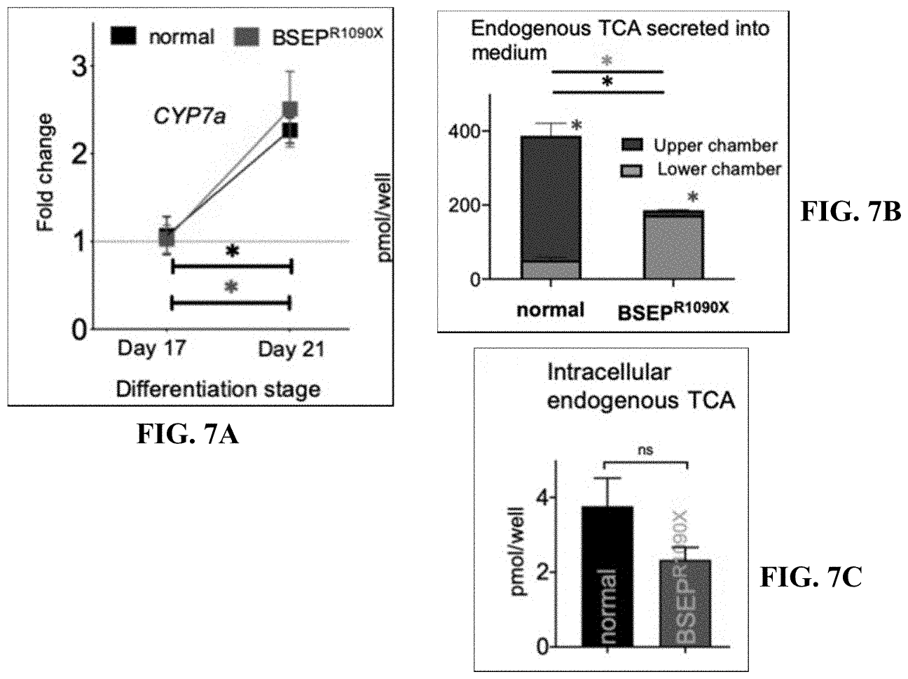

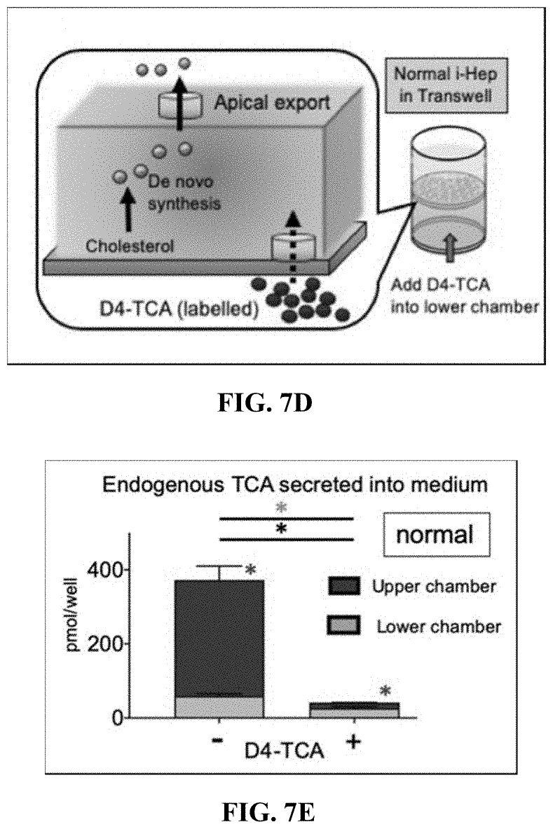

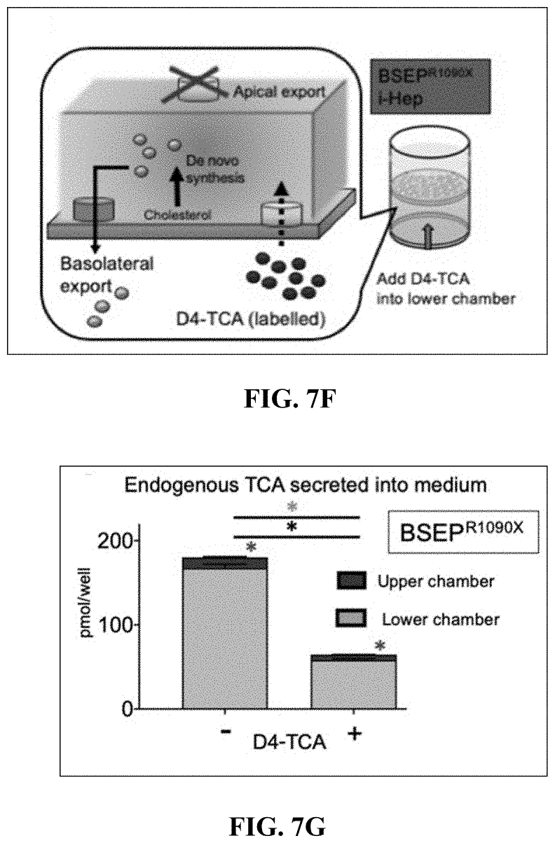

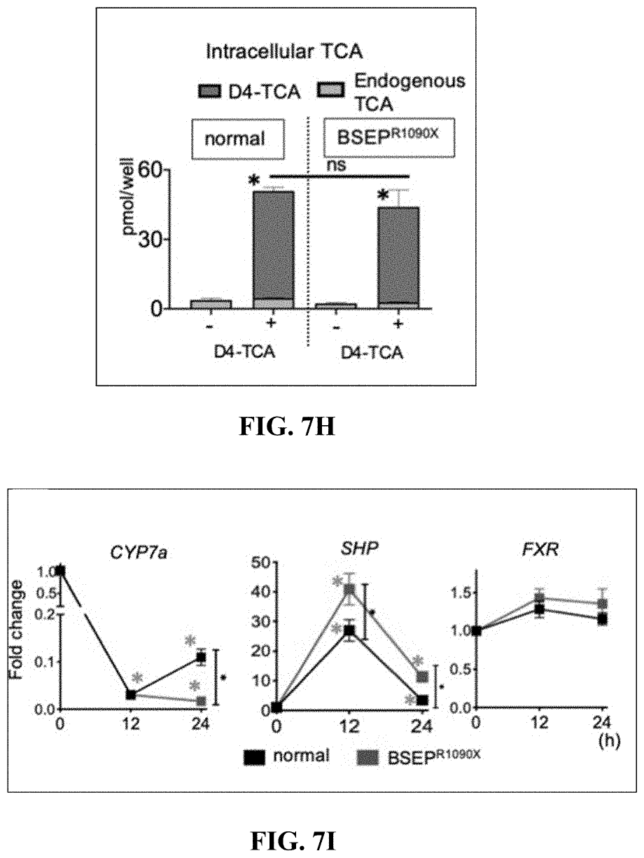

Cholestasis in patients with PFIC2 becomes prominent during the first few weeks after birth as hepatocytes initiate de novo bile acid synthesis. Based on the findings of basolateral export of exogenous bile acids, the fate of intracellular endogenous bile acids synthesized in BSEP deficient hepatocytes was investigated. To determine in which stage the i-Hep culture system induces de novo bile acid synthesis, changes in gene expression of CYP7a by RT-PCR were measured. Both normal and BSEP^{R1090X} i-Hep exhibit minimal expression of CYP7a until day 17 of the differentiation stage, then at the final stage of the differentiation (day 21) CYP7a expression is increased in both normal and BSEP^{R1090X} i-Hep (FIG. 7A). This suggests that i-Hep start synthesizing bile acids de novo at the last stage of the differentiation process.

To assess the impact of truncated BSEP on the export of intracellular bile acids synthesized de novo, the concentration of endogenous TCA secreted into the culture medium from i-Hep was measured (FIG. 7B). After 48 hours of incubation in fresh culture medium, the culture supernatant from the upper chamber and lower chamber were collected separately, as well as the cell lysates. The normal i-Hep exported more TCA into the upper chamber than into the lower chamber. This suggests that normal i-Hep predominantly export TCA via the apical membrane. Consistent with abnormal BSEP function, BSEP^{R1090X} i-Hep exported diminished amount of TCA into the upper chamber but significantly more TCA into the lower chamber, indicating that BSEP^{R1090X} i-Hep predominantly export endogenous TCA via the basolateral membrane. To further determine whether BSEP^{R1090X} i-Hep accumulate endogenous TCA in the cytoplasm, the intracellular amount of TCA in BSEP^{R1090X}_{and} normal i-Hep was measured (FIG. 7C). BSEP^{R1090X} and normal i-Hep showed a comparable amount of intracellular TCA. These data indicate that maturing hepatocytes with BSEP deficiency initiate bile acid export via the basolateral membrane when de novo bile acid synthesis commences, seemingly as an adaptive mechanism to prevent the accumulation of intracellular bile acids.

(viii) Basolateral Transport of Exogenous Bile Acids Suppresses the De Novo Synthesis of Endogenous Bile Acids Via FXR Pathway in BSEP^{R1090X} i-Hep

During trans-hepatocellular transport of the sinusoidal bile acids to the bile canaliculus, de novo bile acid synthesis is suppressed. To determine whether sinusoidal bile acids in the basolateral domain suppress bile acid synthesis in BSEP deficient hepatocytes, de novo bile acid synthesis and transcellular bile acid transport using D4-TCA as an exogenous bile acid were simultaneously quantified (FIGS. 7D and 7F). The exogenous D4-TCA (10 μM) was added in the lower chamber media and was quantified by mass spectrometry, separately from the endogenous TCA. In normal i-Hep, while D4-TCA in the lower chamber was transported to the upper (data not shown), in the same time period, de novo synthesis of TCA by the normal i-Hep was significantly suppressed (FIG. 7E). In contrast, D4-TCA was minimally transported to the upper chamber in BSEP^{R1090X} i-Hep (data not shown), but de novo synthesis of TCA was still significantly suppressed (FIG. 7G). To determine change of the intracellular TCA accumulation by exogenous D4-TCA, endogenous TCA and D4-TCA in the cell lysates after the incubation was measured (FIG. 7H). Normal and BSEP^{R1090X} i-Hep accumulated comparable amount of D4-TCA intracellularly. This result suggests that intracellular TCA, taken up by either normal or BSEP^{R1090X} regulates the rate-limiting step of bile acid synthesis.

To determine whether the regulatory effect was mediated by the FXR pathway, gene expression of FXR and its target genes were quantified, SHP and CYP7a, in i-Hep after exogenous TCA was added in the lower chambers (FIG. 7I). Both normal and BSEP^{R1090X} i-Hep exhibit FXR pathway activation, shown as an increased expression of SHP and decreased expression of CYP7a, when importing TCA. Thus, these results indicate BSEP deficient hepatocytes are able to suppress de novo bile acid synthesis via FXR pathway, when they are not transporting bile acids to the bile canaliculus.

### CONCLUSIONS

Intracellular accumulation of conjugated bile acids in BSEP deficient hepatocytes has been proposed since conjugated bile acids are not excreted in the bile and are found in the liver in high concentration. However, direct evidence of intracellular accumulation of bile acids in human hepatocytes is lacking. In this report, new insights into the mechanism of cellular regulation of intracellular bile acids are provided. By using a newly established in vitro system of human hepatocytes, which recapitulates the expression pattern of truncated BSEP, it was found that hepatocytes with BSEP deficiency in part use basolateral transporters, MRP4, to export conjugated bile acids in order to prevent their intracellular accumulation.

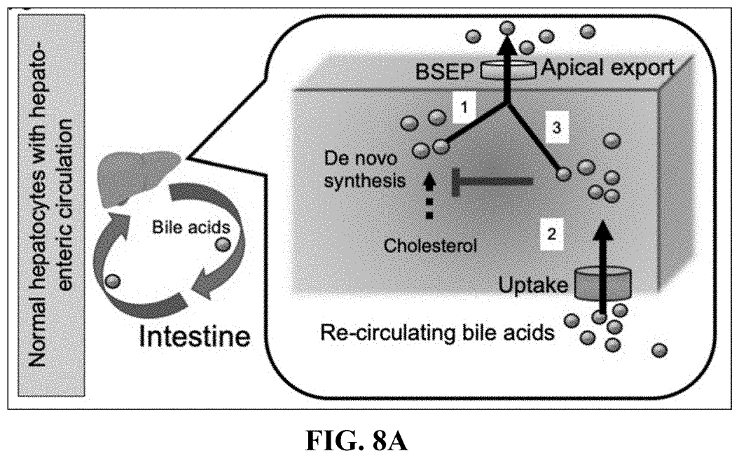

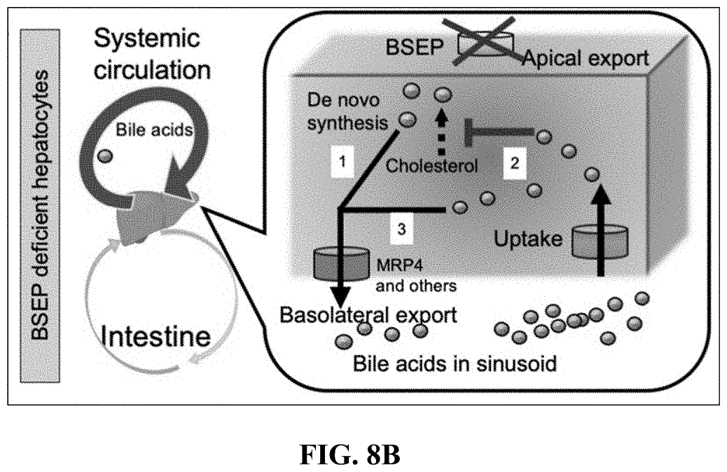

Hepato-enteric bile acid circulation reaches homeostasis by the interaction between transcellular bile acid transport and de novo synthesis mediated by intracellular bile acids in hepatocytes (FIG. 8A). i-Hep in culture system described herein synthesized de novo bile acids at the last stage of the hepatic differentiation under the regulation of HGF, consistent with previous reports of spontaneous bile acid synthesis and secretion by cultured hepatocytes. Ellis et al., Methods Mol Biology Clifton N J 640:417-430 (2010); Liu et al., Toxicol Sci 141:538-546 (2014); and Einarsson et al., World J Gastroentero 6:522-525 (2000). The present study demonstrated that human hepatocytes develop regulatory mechanisms to control the concentration of intracellular conjugated bile acids when BSEP is genomically deficient. The BSEP deficient hepatocytes export endogenous conjugated bile acids via the basolateral membrane as they mature. In patients with PFIC2, since sinusoidal bile acids do not flow into the hepato-enteric circulation, they remain in the systemic circulation, leading to jaundice and cholestasis (FIG. 8B). The mechanisms regulating bile acids accumulating in the systemic circulation and de novo bile acid synthesis have not been defined previously.

In this report, it was demonstrated BSEP deficient hepatocytes are able to down-regulate de novo bile acid synthesis via the uptake and export of bile acids on the basolateral domain, while preventing accumulation of intracellular bile acids. This suggests that BSEP deficient hepatocytes can achieve homeostasis of bile acids concentration of the systemic circulation.

The analysis of ultrastructure showed structural disturbance of the basolateral membrane in BSEP^{R1090X} i Hep. Previous studies showed that increased concentration of bile acids increases lipid fluidity of plasma membrane and disrupt membrane functional domain Scharschmidt et al., Hepatology 1:137-145(1981). It was speculated that constant intracellular-to-basolateral reflux of bile acid may cause abnormally increased concentration of bile acids between the lateral membranes of adjunct cells, thus induce membrane degradation or instability. Given that these changes were found in the liver of patients with PFIC2, they may be important pathophysiological features of BSEP deficiency.

This report provides a proof of concept for a novel in vitro disease model for BSEP deficiency. By generating isogenic iPSCs through CRISPR/Cas9 technology, it was able to elucidate a direct molecular consequence of a single nucleotide variant found in patients. This system allows for directly determination of the cellular and biochemical effect of previously unreported genetic variants and the molecular consequence of missense mutations, often reported as “variant of unknown clinical significance”. As the knowledge of disease-causing variants further accumulates, it would be relied on to predict the clinical course from the genotype and design personalized management strategies at an early stage of the disease.

In summary, these findings reveal novel mechanisms that underlie the pathophysiology of BSEP deficiency and provide targets for therapeutic intervention in patients with PFIC2.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method of generating a population of hepatocyte-like cells, the method comprising:
(i) culturing a population of pluripotent stem cells in an endoderm differentiation medium; wherein the pluripotent stem cells comprise a genetically modified ABCB11 gene;
(ii) culturing a population of cells obtained from step (i) in a hepatic specification medium; and
(iii) culturing a population of cells obtained from step (ii) in a hepatocyte maturation medium to produce a population of hepatocyte-like cells.

2. The method of claim 1, wherein the genetically modified ABCB11 gene express a truncated mutant of a bile salt export pump (BSEP) protein.

3. The method of claim 2, wherein the truncated mutant of the BSEP protein is a R1090X truncation mutant.

4. The method of claim 1, wherein the genetic modification of the ABCB11 gene is performed by CRISPR/Cas9-mediated gene editing.

5. The method of claim 1, wherein the pluripotent stem cells are induced pluripotent stem cells (iPSCs).

6. The method of claim 1, wherein the endoderm differentiation medium comprises:
a. an activin,
b. insulin, and
c. an activator of Wnt signaling pathway, a Rho-associated protein kinase (ROCK) inhibitor, a GSK3 inhibitor, or a combination thereof.

7. The method of claim 6, wherein the endoderm differentiation medium comprises activin, insulin, the activator of Wnt signaling pathway, and the ROCK inhibitor.

8. The method of claim 6, wherein the endoderm differentiation medium comprises activin, insulin, the activator of Wnt signaling pathway, and the inhibitor of class I histone deacetylase.

9. The method of claim 6, wherein the endoderm differentiation medium comprises activin, insulin, the GSK3 inhibitor, and the inhibitor of class I histone deacetylase.

10. The method of claim 6, wherein the endoderm differentiation medium comprises activin, insulin, the GSK3 inhibitor, and the ROCK inhibitor.

11. The method of claim 6, wherein the endoderm differentiation medium comprises an activin, insulin, and the GSK3 inhibitor.

12. The method of claim 6, wherein the inhibitor of class I histone deacetylase is sodium butyrate, wherein the activator of Wnt signaling pathway is Wnt3a, wherein the GSK inhibitor is CHIR99021, and/or wherein the ROCK inhibitor is Y 27632.

13. The method of claim 1, wherein step (i) is performed by culturing the population of pluripotent stem cells in the endoderm differentiation medium for about 5-8 days.

14. The method of claim 1, wherein step (i) is performed by:
(a) culturing the population of pluripotent stem cells in a first endoderm differentiation medium for one day, wherein the first endoderm differentiation medium comprises activin, insulin, the activator of Wnt signaling pathway, and the ROCK inhibitor;
(b) culturing the population of pluripotent stem cells in a second endoderm differentiation medium following step (a) for one day, wherein the second endoderm differentiation medium comprises activin, insulin, the activator of Wnt signaling pathway, and the inhibitor of class I histone deacetylase;
(c) culturing the population of pluripotent stem cells in a third endoderm differentiation medium following step (c) for two days, wherein the third endoderm differentiation medium comprises activin, insulin, the GSK3 inhibitor, and the inhibitor of class I histone deacetylase;
(d) culturing the population of pluripotent stem cells in a fourth endoderm differentiation medium following step (c) for one day, wherein the fourth endoderm differentiation medium comprises activin, insulin, the GSK3 inhibitor, and the ROCK inhibitor; and
(e) culturing the population of pluripotent stem cells in a fifth endoderm differentiation medium following step (d) for one day, wherein the fifth endoderm differentiation medium comprises activin, insulin, and the GSK3 inhibitor.

15. The method of claim 14, wherein after step (c) and prior to step (d), the population of pluripotent stem cells is placed on a permeable membrane.

16. The method of claim 1, wherein in step (i) further comprises culturing the cells in a first cell culture vessel comprising an upper chamber and a lower chamber; wherein both the upper chamber and the lower chamber are separated with a permeable membrane optionally coated with at least one extracellular matrix protein and wherein the cells are in contact with the permeable membrane.

17. The method of claim 16, wherein the cells are first cultured in a second cell culture vessel for about 4 days and then cultured in the first cell culture vessel.

18. The method of claim 17, wherein the first culture vessel, the second culture vessel, or both are coated with at least one extracellular matrix protein.

19. The method of claim 6, wherein the inhibitor of class I deacetylase activity is removed from the medium after about 4 days.

20. The method of claim 1, wherein the hepatic specification medium comprises:
a. a fibroblast growth factor (FGF), and
b. a bone morphogenic protein (BMP).

21. The method of claim 20, wherein (a) is FGF2 and/or wherein (b) is BMP4.

22. The method of claim 1, wherein step (ii) is performed by culturing the population of cells from step (i) in the hepatic specification medium for about 3 days.

23. The method of claim 1, wherein the hepatocyte maturation medium comprises a hepatocyte growth factor (HGF) and is free of a human epidermal growth factor (EGF).

24. The method of claim 23, wherein the hepatocyte maturation medium further comprises transferrin, dexamethasone, hydrocortisone, and insulin.

25. The method of claim 1, wherein step (iii) comprises culturing the population of cells from step (ii) on a permeable membrane in a cell culture vessel.

26. The method of claim 25, wherein the cell culture comprises an upper chamber and a lower chamber; wherein both the upper chamber and the lower chamber are separated with the permeable membrane and wherein the cells are placed on the permeable membrane.

27. The method of claim 25, wherein the permeable membrane is coated with at least one extracellular matrix protein.

28. The method of claim 1, wherein step (iii) is performed by culturing the population of cells from step (ii) for about 10-14 days.

29. The method of claim 1, wherein step (iii) is performed in the absence of human umbilical vein endothelial cells (HUVEC) and/or mesenchymal stem cells (MSC).

30. A population of hepatocyte-like cells, which is produced by a method of claim 1.

31. The population of hepatocyte-like cells of claim 30, which form apico-basolateral polarity.

32. An in vitro cell culture system, comprising:
(i) a cell culture vessel comprising an upper chamber and a lower chamber; wherein both the upper chamber and the lower chamber comprise a medium for culturing hepatocytes;
a permeable membrane separating the upper chamber and the lower chamber; and
(iii) a layer of hepatocyte-like cells grown on the permeable membrane, wherein the hepatocyte-like cells are differentiated from a population of pluripotent stem cells having a modified ABCB11 gene.

33. The in vitro cell culture system of claim 31, wherein the hepatocyte-like cells are generated by a method comprising:
(i) culturing a population of pluripotent stem cells in an endoderm differentiation medium; wherein the pluripotent stem cells comprise a genetically modified ABCB11 gene;
(ii) culturing a population of cells obtained from step (i) in a hepatic specification medium; and
(iii) culturing a population of cells obtained from step (ii) in a hepatocyte maturation medium to produce a population of hepatocyte-like cells.

34. A method for identifying an agent for treating a cholestatic liver disease, the method comprising:
(i) providing an in vitro cell culture system set forth in claim 31,
(ii) adding a bile acid to the lower chamber,
(iii) culturing the hepatocyte-like cells in the presence of a candidate agent;
(iv) measuring the concentration of the bile acid in the upper chamber and/or in the lower chamber; and
(v) identifying the candidate agent as an agent for treating a cholestatic liver disease, if the candidate agent changes the bile acid concentration determined in step (iv) as compared with the in vitro cell culture system in the absence of the candidate agent.

35. A method of generating a population of hepatocyte-like cells, the method comprising:
(i) culturing a population of pluripotent stem cells in an endoderm differentiation medium;
(ii) culturing a population of cells obtained from step (i) in a hepatic differentiation medium; and
(iii) culturing a population of cells obtained from step (ii) in a hepatocyte maturation medium, wherein step (iii) is performed in the absence of human umbilical vein endothelial cells (HUVEC) and/or mesenchymal stem cells (MSC) to produce a population of hepatocyte-like cells.

36. The method of claim 35, wherein the endoderm differentiation medium comprises:
a. an activin,
b. insulin,
c. an inhibitor of class I histone deacetylase, an activator of Wnt signaling pathway, a Rho-associated protein kinase (ROCK) inhibitor, a GSK3 inhibitor, or a combination thereof.

37. The method of claim 36, wherein the endoderm differentiation medium comprises activin, insulin, the activator of Wnt signaling pathway, and the ROCK inhibitor.

38. The method of claim 36, wherein the endoderm differentiation medium comprises activin, insulin, the activator of Wnt signaling pathway, and the inhibitor of class I histone deacetylase.

39. The method of claim 36, wherein the endoderm differentiation medium comprises activin, insulin, the GSK3 inhibitor, and the inhibitor of class I histone deacetylase.

40. The method of claim 36, wherein the endoderm differentiation medium comprises activin, insulin, the GSK3 inhibitor, and the ROCK inhibitor.

41. The method of claim 36, wherein the endoderm differentiation medium comprises activin, insulin, and the GSK3 inhibitor.

42. The method of claim 36, wherein the inhibitor of class I histone deacetylase is sodium butyrate, wherein the activator of Wnt signaling pathway is Wnt3a, wherein the GSK inhibitor is CHIR99021, and/or wherein the ROCK inhibitor is Y 27632.

43. The method of claim 35, wherein step (i) is performed by culturing the population of pluripotent stem cells in the endoderm differentiation medium for about 5-8 days.

44. The method of claim 35, wherein step (i) is performed by:
(a) culturing the population of pluripotent stem cells in a first endoderm differentiation medium for one day, wherein the first endoderm differentiation medium comprises activin, insulin, the activator of Wnt signaling pathway, and the ROCK inhibitor;
(b) culturing the population of pluripotent stem cells in a second endoderm differentiation medium following step (a) for one day, wherein the second endoderm differentiation medium comprises activin, insulin, the activator of Wnt signaling pathway, and the inhibitor of class I histone deacetylase;
(c) culturing the population of pluripotent stem cells in a third endoderm differentiation medium following step (c) for two days, wherein the third endoderm differentiation medium comprises activin, insulin, the GSK3 inhibitor, and the inhibitor of class I histone deacetylase;
(d) culturing the population of pluripotent stem cells in a fourth endoderm differentiation medium following step (c) for one day, wherein the fourth endoderm differentiation medium comprises activin, insulin, the GSK3 inhibitor, and the ROCK inhibitor; and
(e) culturing the population of pluripotent stem cells in a fifth endoderm differentiation medium following step (d) for one day, wherein the fifth endoderm differentiation medium comprises activin, insulin, and the GSK3 inhibitor.

45. The method of claim 44, wherein after step (c) and prior to step (d), the population of pluripotent stem cells is placed on a permeable membrane.

46. The method of claim 35, wherein in step (i) further comprises culturing the cells in a first cell culture vessel comprising an upper chamber and a lower chamber; wherein both the upper chamber and the lower chamber are separated with a permeable membrane optionally coated with at least one extracellular matrix protein and wherein the cells are in contact with the permeable membrane.

47. The method of 46, wherein the cells are first cultured in a second cell culture vessel for about 4 days and then cultured in the first cell culture vessel.

48. The method of claim 47, wherein the first culture vessel, the second culture vessel, or both are coated with at least one extracellular matrix protein.

49. The method of claim 36, wherein the inhibitor of class I deacetylase activity is removed from the medium after about 4 days.

50. The method of claim 35, wherein the hepatic specification medium comprises:
a. a fibroblast growth factor (FGF), and
b. a bone morphogenic protein (BMP).

51. The method of claim 50, wherein (a) is FGF2 and/or wherein (b) is BMP4.

52. The method of claim 35, wherein step (ii) is performed by culturing the population of cells from step (i) in the hepatic specification medium for about 3 days.

53. The method of claim 35, wherein the hepatocyte maturation medium comprises a hepatocyte growth factor (HGF) and is free of a human epidermal growth factor (EGF).

54. The method of claim 53, wherein the hepatocyte maturation medium further comprises transferrin, hydrocortisone, and insulin.

55. The method of claim 35, wherein step (ii) and (iii) comprises culturing the population of cells from step (ii) on a permeable membrane in a cell culture vessel.

56. The method of claim 55, wherein the cell culture comprises an upper chamber and a lower chamber; wherein both the upper chamber and the lower chamber are separated with the permeable membrane and wherein the cells are placed on the permeable membrane.

57. The method of claim 56, wherein the permeable membrane is coated with at least one extracellular matrix protein.

58. The method of claim 35, wherein step (iii) is performed by culturing the population of cells from step (ii) for about 10-14 days.

59. A population of hepatocyte-like cells, which is produced by a method of claim 35.

60. A method for identifying an agent which disrupts bile acid transport and/or synthesis, the method comprising:
(i) providing an in vitro cell culture system;
(ii) adding a bile acid to the lower chamber;
(iii) culturing the hepatocyte-like cells in the presence of a candidate agent;
(iv) measuring the concentration of the bile acid in the upper chamber and/or in the lower chamber; and
(v) identifying the candidate agent as an agent which disrupts bile acid transport and/or synthesis, if the candidate agent changes the bile acid concentration determined in step (iv) as compared with the in vitro cell culture system in the absence of the candidate agent;
wherein the in vitro cell culture system comprises (a) a cell culture vessel comprising an upper chamber and a lower chamber; wherein both the upper chamber and the lower chamber comprise a medium for culturing hepatocytes; (b) a permeable membrane separating the upper chamber and the lower chamber; and (c) a layer of hepatocyte-like cells grown on the permeable membrane, wherein the hepatocyte-like cells have a functional apico-basolateral polarity, transport of bile acids and/or de novo synthesis of bile acids prior to the addition of the candidate agent.

61. The method of claim 60, wherein the hepatocyte-like cells are a population of cells produced by a method comprising:
(i) culturing a population of pluripotent stem cells in an endoderm differentiation medium;
(ii) culturing a population of cells obtained from step (i) in a hepatic differentiation medium; and
(iii) culturing a population of cells obtained from step (ii) in a hepatocyte maturation medium, wherein step (iii) is performed in the absence of human umbilical vein endothelial cells (HUVEC) and/or mesenchymal stem cells (MSC) to produce a population of hepatocyte-like cells.


SEQUENCE LISTING (2 sequences)

SEQ ID NO: 1 - DNA, 12 bp, Homo sapiens
ccttctcgacct

SEQ ID NO: 2 - DNA, 12 bp, Homo sapiens
ccttcatgacca